(19) United States
(12) Patent Application Publication
(10) Pub. No.: US 2025/0207090 A1
(43) Pub. Date: Jun. 26, 2025

(54) METHODS AND COMPOSITIONS FOR PRODUCING OOGONIA-LIKE CELLS

(71) Applicant: President and Fellows of Harvard College, Cambridge, MA (US)

(72) Inventors: Christian Kramme, Cambridge, MA (US); Pranam Chatterjee, Cambridge, MA (US); Merrick Pierson Smela, Cambridge, MA (US); George M. Church, Cambridge, MA (US)

(73) Assignee: President and Fellows of Harvard College, Cambridge, MA (US)

(21) Appl. No.: 18/852,057

(22) PCT Filed: Mar. 30, 2023
(86) PCT No.: PCT/US2023/065145

§ 371 Date: Sep. 27, 2024

Related U.S. Application Data
(60) Provisional application No. 63/326,607, filed on Apr. 1, 2022.

(51) Int. Cl. C12N 5/075; C12N 15/85

(52) U.S. Cl. CPC C12N 5/0609; C12N 15/85; C12N 2501/11; C12N 2501/125; C12N 2501/155; C12N 2501/16; C12N 2506/45; C12N 2533/90; C12N 2800/107; C12N 2830/002

(57) ABSTRACT

Provided herein are methods and compositions for differentiating induced pluripotent stem cells into oogonia-like cells by overexpressing transcription factors such as ZNF281, LHX8, and/or SOHLH1.

### CROSS-REFERENCE TO RELATED APPLICATIONS

This application claims the benefit under 35 U.S.C. § 119 (e) of U.S. Provisional Application No. 63/326,607, filed Apr. 1, 2022, which is hereby incorporated by reference in its entirety.

### REFERENCE TO AN ELECTRONIC SEQUENCE LISTING

The contents of the electronic sequence listing (H049870757WO00-SEQ-KVC.xml; Size: 12,492 bytes; and Date of Creation: Mar. 28, 2023) is herein incorporated by reference in its entirety.

### BACKGROUND

Oogonia are specialized cells that, upon maturation, form primordial follicles in the female fetus. Oogonia proliferate through mitosis before differentiating into oocytes that participate in sexual reproduction. Oogonia dysfunction forms the basis of many forms of human female infertility, yet efficient methods for generating oogonia in vitro remain elusive.

### SUMMARY

The present disclosure relates, at least in part, to methods and compositions for generating oogonia in vitro from pluripotent stem cells (PSCs). The present disclosure provides experimental data demonstrating, unexpectedly, that overexpression of certain transcription factors, for example, Zinc Finger Protein 281 (ZNF281), LIM Homeobox 8 (LHX8), and Spermatogenesis and Oogenesis Specific Basic Helix-Loop-Helix 1 (SOHLH1), is sufficient to generate oogonia (e.g., DDX4+ oogonia-like cells) from PSCs in as few as four days.

Some aspects of the present disclosure provide a pluripotent stem cell (PSC) comprising: an engineered polynucleotide comprising an open reading frame encoding a protein selected from ZNF281, LHX8, and SOHLH1.

In some embodiments, the PSC comprises the engineered polynucleotide comprising an open reading frame encoding ZNF281.

In some embodiments, the PSC comprises the engineered polynucleotide comprising an open reading frame encoding LHX8.

In some embodiments, the PSC comprises the engineered polynucleotide comprising an open reading frame encoding SOHLH1.

In some embodiments, the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.

In some embodiments, the PSC further comprises an engineered polynucleotide comprising an open reading frame encoding a Folliculogenesis Specific BHLH Transcription Factor (FIGLA) protein, optionally wherein the PSC expresses or overexpresses FIGLA.

In some embodiments, the PSC further comprises: an engineered polynucleotide comprising an open reading frame encoding a Distal-Less Homeobox 5 (DLX5) protein; and an engineered polynucleotide comprising an open reading frame encoding an Hematopoietically Expressed Homeobox (HHEX) protein, optionally wherein the PSC expresses or overexpresses the DLX5 protein and the HHEX protein.

In some embodiments, the PSC further comprises: an engineered polynucleotide comprising an open reading frame encoding a DEAD-Box Polypeptide-4 (DDX4) protein; an engineered polynucleotide comprising an open reading frame encoding a Deleted in AZoospermia (DAZL) protein; and an engineered polynucleotide comprising an open reading frame encoding a Boule Homolog (BOLL) protein, optionally wherein the PSC expresses or overexpresses the DDX4 protein, the DAZL protein, and the BOLL protein.

In some embodiments, the open reading frame of the engineered polynucleotide is operably linked to a heterologous promoter.

In some embodiments, the heterologous promoter is an inducible promoter.

Other aspects of the present disclosure provide a PSC comprising: a protein selected from ZNF281, LHX8, and SOHLH1, wherein the protein is overexpressed.

In some embodiments, the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.

In some embodiments, the PSC further comprises a FIGLA protein, optionally wherein the PSC expresses or overexpresses FIGLA.

In some embodiments, the PSC further comprises: a DLX5 protein and an HHEX protein, optionally wherein the PSC expresses or overexpresses the DLX5 protein and the HHEX protein.

In some embodiments, the PSC further comprises: a DDX4 protein, a DAZL protein, and a BOLL protein, optionally wherein the PSC expresses or overexpresses the DDX4 protein, the DAZL protein, and the BOLL protein.

In some embodiments, the PSC is a human PSC.

In some embodiments, the PSC is an induced PSC (iPSC).

In some embodiments, the PSC comprises 1-20 copies of the engineered polynucleotide comprising the open reading frame encoding the protein selected from ZNF281, LHX8, and SOHLH1. In some embodiments, the PSC comprises 8-10 copies of the engineered polynucleotide comprising the open reading frame encoding the protein selected from ZNF281, LHX8, and SOHLH1.

Some aspects of the present disclosure provide a composition comprising: a population of the PSC of any one of the preceding paragraphs or described elsewhere herein. In some embodiments, the population comprises at least 2500/cm² of the PSC.

Other aspects of the present disclosure provide a method, comprising: culturing, in culture media, a population of pluripotent stem cells (PSCs) to produce an expanded population of PSCs; and expressing in PSCs of the expanded population a protein selected from ZNF281, LHX8, and SOHLH1 to produce oogonia-like cells.

In some embodiments, the PSCs of the expanded population comprise an engineered polynucleotide comprising an open reading frame encoding ZNF281.

In some embodiments, the PSCs of the expanded population comprise an engineered polynucleotide comprising an open reading frame encoding LHX8.

In some embodiments, the PSCs of the expanded population comprise an engineered polynucleotide comprising an open reading frame encoding SOHLH1.

In some embodiments, the PSCs of the expanded population further comprise an engineered polynucleotide comprising an open reading frame encoding a FIGLA protein.

In some embodiments, the PSCs of the expanded population further comprise: an engineered polynucleotide comprising an open reading frame encoding a DLX5 protein; and an engineered polynucleotide comprising an open reading frame encoding an HHEX protein.

In some embodiments, n the PSCs of the expanded population further comprise: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein.

In some embodiments, the open reading frame of the engineered polynucleotide is operably linked to a heterologous promoter.

In some embodiments, the heterologous promoter is an inducible promoter.

In some embodiments, the population comprises 1×10²-1×10⁷ PSCs.

In some embodiments, the population of PSCs is cultured for about 3-5 days, optionally about 4 days.

In some embodiments, the oogonia-like cells are DDX4⁺.

In some embodiments, the oogonia-like cells independently have a diameter of about 20 micrometers to 180 micrometers.

Some aspects of the present disclosure provide a method comprising: (a) delivering to PSCs an engineered polynucleotide comprising an inducible promoter operably linked to an open reading frame encoding a protein selected from ZNF281, LHX8, and SOHLH1; (b) culturing the PSCs in feeder-free, serum-free culture media to produce an expanded population of PSCs; and (c) culturing PSCs of the expanded population in a series of induction media comprising an inducing agent to produce DDX4+ oogonia-like cells.

In some embodiments, the engineered polynucleotide is a transposon and the delivering further comprises delivering a transposase to the PSCs.

In some embodiments, the inducible promoter is a chemically-inducible promoter, optionally a doxycycline-inducible promoter.

In some embodiments, the feeder-free, serum-free culture media of (b) comprises a solubilized basement membrane preparation extracted from the Engelbreth-Holm-Swarm (EHS) mouse sarcoma.

In some embodiments, the solubilized basement membrane preparation comprises extracellular matrix (ECM) proteins and growth factors.

In some embodiments, the ECM proteins are selected from Laminin, Collagen IV, heparan sulfate proteoglycans, and entactin/nidogen.

In some embodiments, the feeder-free, serum-free culture media of (b) comprises growth factors selected from recombinant human basic fibroblast growth factor (rh bFGF) and recombinant human transforming growth factor β (rh TGFβ).

In some embodiments, the culturing of (b) is for about 6 to about 24 hours.

In some embodiments, the PSCs of the expanded population of (c) are cultured at a density of about 2,000 cells/cm² to about 3,000 cells/cm².

In some embodiments, the culturing of (c) comprises culturing the PSCs is a first induction media, culturing the PSCs in a second induction media, culturing the PSCs in a third induction media, and culturing the PSCs in a fourth induction media.

In some embodiments, the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.

In some embodiments, the second induction media comprises one or more of B-27, an inducing agent (e.g., doxycycline), a small molecule inhibitor of tankyrase (TNKS), and a human bone morphogenic protein 4 (hBMP4).

In some embodiments, the third induction media comprises one or more of B-27, an inducing agent (e.g., doxycycline), a small molecule inhibitor of tankyrase, stem cell factor (SCF), and epidermal growth factor (EGF).

In some embodiments, the fourth induction media comprises one or more of B-27, an inducing agent (e.g., doxycycline), a small molecule inhibitor of tankyrase, hBMP4, SCF, and EGF.

Some aspects of the present disclosure provide an oogonia-like cell produced by the method of any one of the preceding paragraphs or described elsewhere herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows oogenic scoring of TFs from scRNA-Seq, in which TF enrichment is scored based on presence of TFs in cells that express oocyte marker genes.

FIGS. 2A-2C show that three TFs drive high DDX4 expression when induced during germ cell formation. FIG. 2A shows flow cytometry data of DDX4 expression in cells expressing no TFs (top left), ZNF281 (top right), SOHLH1 (bottom left) and LHX8 (bottom right). FIG. 2B shows geometric mean expression of DDX4 fluorescent intensity compared to controls. FIG. 2C shows flow cytometry data of DDX4 expression in cells expressing the D3 combination, showing high DDX4 oogonia-like characteristics.

FIGS. 3A-3B show that the D3 TF combination drives formation of cells with oocyte-like transcriptomes. FIG. 3A shows RNA-Seq gene expression patterns, shown as a log 2 fold change compared to human induced pluripotent stem cell (hiPSC) controls, for select oogenesis signature genes. FIG. 3B shows a cell type classification analysis (TROM) in which the transcriptome of the D3 oogonia like cells are compared to a reference ovarian atlas, showing statistically significant similarity (defined as greater than TROM=12) to in vivo antral and secondary oocytes.

FIG. 4 shows a schematic representation of a method for high DDX4 expressing cell formation from stem cells.

FIG. 5 shows testing of combinations of TFs for oogonia formation. The percentage of cells that express DDX4 and the percentage of cells that express NPM2 are shown.

### DETAILED DESCRIPTION

Oogonia are essential cells involved in reproduction. Oogonia dysfunction forms the basis of many forms of human female infertility (Garg et al. 2015). Accordingly, there is an unmet need to develop methods for generating oogonia and oogonia-like cells. Such cells may form the basis of a therapeutic intervention for human female infertility, for example. It should be understood that the term “oogonia-like cell” encompasses cells that express oogonia-specific markers, such as DDX4, and exhibit other characteristics of naturally-occurring oogonia cells.

Current methods for generating DDX4-positive human oogonia-like cells in vitro, for example, from human induced pluripotent stem cells (hiPSCs), are laborious, expensive and time consuming. Current methods utilize co-culture of induced human primordial germ cell-like cells (hPGCLCs) and mouse E12.5 fetal gonad cells (mFGCs) to form xenogeneic reconstituted ovaries (xrOvaries), which when grown over ˜120 days induce a small population of human DDX4+ oogonia-like cells (Yamashiro et al. 2018). This approach, while able to make DDX4+ oogonia-like cells, requires extensive technical expertise, expensive culturing techniques, and lengthy differentiation periods, which render the method intractable for most high throughput applications. Aspects of the present disclosure relate to a method of using direct transcription factor overexpression in conjunction with growth factor culturing to induce DDX4+ oogonia-like cells from stem cells in 4 days.

### Oogonia-Like Cells

Some aspects of the present disclosure provide oogonia-like cells and methods of producing such cells. Oogonia are small diploid cells, which, upon maturation, form primordial follicles in female fetuses. Oogonia are formed in large numbers by mitosis early in fetal development from primordial germ cells. During human development, primordial germ cells differentiate into oogonia, which further proliferate via mitosis. Following proliferation, oogonia differentiate into primary oocytes through asymmetric division. One daughter cell produced through asymmetric division of primary oocytes becomes an oocyte through the process of oogenesis. Oocytes produced through oogenesis eventually undergo meiosis and participate in sexual reproduction (Sathananthan et al. 2006).

Oogonia and oocytes produced through oogenesis express the gene DEAD-Box Polypeptide-4 (DDX4) (e.g., UniProtKB-Q9NQI0 (DDX4_HUMAN)), a putative marker gene for female germ cells (Danny et al. 2021). Thus, in some embodiments, the oogonia-like cells produced by the methods provided herein are DDX4+ oogonia-like cells (i.e., cells that express DHX4 protein).

There are other characteristics of oogonia-like cells that distinguish them from non-oogonia-like cells including, but not limited to, size. For example, an oogonia-like cell may have a diameter of about 20 micrometers (μm) to about 180 μm. In some embodiments, an oogonia-like cell has a diameter of about 20 μm to about 160 μm, about 20 μm to about 140 μm, about 20 μm to about 120 μm, about 20 μm to about 100 μm, about 40 μm to about 180 μm, about 40 μm to about 160 μm, about 40 μm to about 140 μm, about 40 μm to about 120 μm, about 40 μm to about 100 μm, about 60 μm to about 180 μm, about 60 μm to about 160 μm, about 60 μm to about 140 μm, about 60 μm to about 120 μm, about 60 μm to about 100 μm, about 80 μm to about 180 μm, about 80 μm to about 160 μm, about 80 μm to about 140 μm, about 80 μm to about 120 μm, about 80 μm to about 100 μm, about 100 μm to about 180 μm, about 100 μm to about 160 μm, about 100 μm to about 140 μm, or about 100 μm to about 120 μm.

### Pluripotent Stem Cells

The oogonia-like cells provided herein are differentiated from pluripotent stem cells. Pluripotent stem cells are cells that have the capacity to self-renew by dividing, and to develop into the three primary germ cell layers of the early embryo (e.g., ectoderm, endoderm, and mesoderm), and therefore into all cells of the adult body, but not extra-embryonic tissues such as the placenta (Shi et al. 2017).

Non-limiting examples of pluripotent stem cells include induced pluripotent cell (iPSCs), “true” embryonic stem cell (ESCs) derived from embryos, embryonic stem cells made by somatic cell nuclear transfer (ntESCs), and embryonic stem cells from unfertilized eggs (parthenogenesis embryonic stem cells, or pESCs). In some embodiments, a pluripotent cell is a human pluripotent cell.

In some embodiments, a pluripotent stem cell is an embryonic stem cell, such as a human embryonic stem cell. “Embryonic stem cell” is a general term for pluripotent stem cells that are made using embryos or eggs, rather than for cells genetically reprogrammed from the body. As used herein, “ESCs” encompass true ESCs, ntESCs, and pESCs.

In other embodiments, a pluripotent stem cell is an induced pluripotent stem cell, such as a human induced pluripotent stem cell. iPSCs may be derived from skin or blood cells that have been reprogrammed back into an embryonic-like pluripotent state that enables the development of an unlimited source of any type of human cell.

Some aspects of the present disclosure provide a PSC comprising: a protein selected from ZNF281, LHX8, and SOHLH1, wherein the protein is expressed or overexpressed. In some embodiments, the protein is expressed at a level that is at least 5%, at least 10%, at least 15%, at least 20%, at least 25%, at least 50%, or at least 100% higher than a control level. In some embodiments, a control level is an endogenous level of the protein, for example in a naturally-occurring pluripotent stem cell. In some embodiments, a PSC comprises ZNF281. In some embodiments, a PSC expresses or overexpresses ZNF281. In some embodiments, a PSC comprises LHX8. In some embodiments, a PSC expresses or overexpresses LHX8. In some embodiments, a PSC comprises SOHLH1. In some embodiments, a PSC expresses or overexpresses SOHLH1.

Data provided herein shows that combinatorial expression of two proteins selected from ZNF281, LHX8, and SOHLH1 results in a 10-50 fold increase in efficiency of DDX4+ oogonia cell-like production, relative to a control, optionally wherein the control is efficiency of DDX4+ oogonia cell-like production in a PSC expressing only one of ZNF281, LHX8, or SOHLH1. In some embodiments, a PSC comprises ZNF281 and LHX8. In some embodiments, a PSC expresses or overexpresses ZNF281 and LHX8. In some embodiments, a PSC comprises ZNF281 and SOHLH1. In some embodiments, a PSC expresses or overexpresses ZNF281 and SOHLH1. In some embodiments, a PSC comprises LHX8 and SOHLH1. In some embodiments, a PSC expresses or overexpresses LHX8 and SOHLH1.

Data provided herein also shows that combinatorial expression of ZNF281, LHX8, and SOHLH1 in PSCs results in a 100-1000 fold increase in efficiency of DDX4+ oogonia cell-like production, relative to a control, optionally wherein the control is efficiency of DDX4+ oogonia cell-like production in a PSC expressing only one of ZNF281, LHX8, or SOHLH1. In some embodiments, a PSC comprises ZNF281, LHX8, and SOHLH1. In some embodiments, a PSC expresses or overexpresses ZNF281, LHX8, and SOHLH1.

Data provided herein further shows that combinatorial expression of FIGLA, ZNF281, LHX8, and SOHLH1 in PSCs results in a ˜10 fold increase in efficiency of DDX4+ oogonia cell-like production, relative to a control, optionally wherein the control is efficiency of DDX4+ oogonia cell-like production in a PSC expressing only ZNF281, LHX8, and SOHLH1. In some embodiments, the cells produced express NPM2, an oocyte-like marker. In some embodiments, a PSC further comprises FIGLA. In some embodiments, a PSC further expresses or overexpresses FIGLA.

Data provided herein further shows that combinatorial expression of FIGLA, DLX5, HHEX, ZNF281, LHX8, and SOHLH1 in PSCs results in an increase in efficiency of DDX4+ oogonia cell-like production, relative to a control, optionally wherein the control is efficiency of DDX4+ oogonia cell-like production in a PSC expressing only ZNF281, LHX8, and SOHLH1. In some embodiments, a PSC further comprises DLX5. In some embodiments, a PSC further expresses or overexpresses DLX5. In some embodiments, a PSC further comprises HHEX. In some embodiments, a PSC further expresses or overexpresses HHEX.

Data provided herein further shows that combinatorial expression of DDX4, DAZL, BOLL, FIGLA, DLX5, HHEX, ZNF281, LHX8, and SOHLH1 in PSCs results in an increase in efficiency of DDX4+ oogonia cell-like production, relative to a control, optionally wherein the control is efficiency of DDX4+ oogonia cell-like production in a PSC expressing only ZNF281, LHX8, and SOHLH1. In some embodiments, a PSC further comprises DDX4. In some embodiments, a PSC further expresses or overexpresses DDX4. In some embodiments, a PSC further comprises DAZL. In some embodiments, a PSC further expresses or overexpresses DAZL. In some embodiments, a PSC further comprises BOLL. In some embodiments, a PSC further expresses or overexpresses BOLL.

Surprisingly, combinatorial expression of ZNF281, LHX8, and SOHLH1 and only one of HHEX, DLX5, DAZL, DDX4, BOLL, or DAZL showed no increase in efficiency of DDX4+ oogonia cell-like production in a PSC.

### Transcription Factors

The oogonia-like cells provided herein are differentiated from pluripotent stem cells, in some embodiments, by expressing one or more (e.g., 2, 3, 4, 5, 6, 7, 8, or 9) transcription factors (i.e., a protein that controls the rate of transcription). Differentiation is the process by which an uncommitted cell or a partially committed cell commits to a specialized cell fate. Aspects of the present disclosure relate to the differentiation of uncommitted pluripotent stem cells into an oogonia-like cell fate. In some embodiments, the transcription factors are selected from ZNF281, LHX8, and SOHLH1. In some embodiments, pluripotent stem cells, such as hPSCs or hiPSCs, are engineered to express or overexpress ZNF281. In some embodiments, pluripotent stem cells, such as hPSCs or hiPSCs, are engineered to express or overexpress LHX8. In some embodiments, pluripotent stem cells, such as hPSCs or hiPSCs, are engineered to express or overexpress SOHLH1. In some embodiments, pluripotent stem cells, such as hPSCs or hiPSCs, are engineered to express or overexpress ZNF281 and LHX8. In some embodiments, pluripotent stem cells, such as hPSCs or hiPSCs, are engineered to express or overexpress ZNF281 and SOHLH1. In some embodiments, pluripotent stem cells, such as hPSCs or hiPSCs, are engineered to express or overexpress LHX8 and SOHLH1. In some embodiments, pluripotent stem cells, such as hPSCs or hiPSCs, are engineered to express or overexpress ZNF281, LHX8, and SOHLH1.

A cell “expressed” a particular protein if the level of the protein in the cell is detectable (e.g., using a known protein assay). A cell “overexpresses” a particular protein (e.g., engineered polynucleotide encoding the protein) if the level of the protein is higher than (e.g., at least 5%, at least 10%, or at least 20% higher than) the level of the protein expressed from an endogenous, naturally-occurring polynucleotide encoding the protein.

In some embodiments, the transcription factors are selected from FIGLA, DLX5, and HHEX. In some embodiments, pluripotent stem cells, such as hPSCs or hiPSCs, are engineered to express or overexpress FIGLA. In some embodiments, pluripotent stem cells, such as hPSCs or hiPSCs, are engineered to express or overexpress DLX5. In some embodiments, pluripotent stem cells, such as hPSCs or hiPSCs, are engineered to express or overexpress HHEX. In some embodiments, pluripotent stem cells, such as hPSCs or hiPSCs, are engineered to express or overexpress FIGLA and DLX5. In some embodiments, pluripotent stem cells, such as hPSCs or hiPSCs, are engineered to express or overexpress FIGLA and HHEX. In some embodiments, pluripotent stem cells, such as hPSCs or hiPSCs, are engineered to express or overexpress DLX5 and HHEX. In some embodiments, pluripotent stem cells, such as hPSCs or hiPSCs, are engineered to express or overexpress FIGLA, DLX5, and HHEX. In some embodiments, pluripotent stem cells, such as hPSCs or hiPSCs, are engineered to express or overexpress ZNF281, LHX8, SOHLH1, and FIGLA. In some embodiments, pluripotent stem cells, such as hPSCs or hiPSCs, are engineered to express or overexpress ZNF281, LHX8, SOHLH1, FIGLA, DLX5, and HHEX.

In some embodiments, pluripotent stem cells, such as hPSCs or hiPSCs, are engineered to express or overexpress DDX4. In some embodiments, pluripotent stem cells, such as hPSCs or hiPSCs, are engineered to express or overexpress DAZL. In some embodiments, pluripotent stem cells, such as hPSCs or hiPSCs, are engineered to express or overexpress BOLL. In some embodiments, pluripotent stem cells, such as hPSCs or hiPSCs, are engineered to express or overexpress DDX4 and DAZL. In some embodiments, pluripotent stem cells, such as hPSCs or hiPSCs, are engineered to express or overexpress DDX4 and BOLL. In some embodiments, pluripotent stem cells, such as hPSCs or hiPSCs, are engineered to express or overexpress DAZL and BOLL. In some embodiments, pluripotent stem cells, such as hPSCs or hiPSCs, are engineered to express or overexpress DDX4, DAZL, and BOLL.

In some embodiments, pluripotent stem cells, such as hPSCs or hiPSCs, are engineered to express or overexpress ZNF281, LHX8, SOHLH1, FIGLA, DLX5, HHEX, DDX4, DAZL, and BOLL.

### Engineered Polynucleotides and Polypeptides

The pluripotent stem cells of the present disclosure, in some embodiments, comprise engineered polynucleotides. An engineered polynucleotide is a nucleic acid (e.g., at least two nucleotides covalently linked together, and in some instances, containing phosphodiester bonds, referred to as a phosphodiester backbone) that does not occur in nature. Engineered polynucleotides include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered polynucleotide may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a polynucleotide is a complementary DNA (cDNA). cDNA is synthesized from a single-stranded RNA (e.g., messenger RNA (mRNA) or microRNA (miRNA)) template in a reaction catalyzed by reverse transcriptase.

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered polynucleotides may be used in accordance with the present disclosure.

In some embodiments, an engineered polynucleotide comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some embodiments, the inducible promoter is a tetracycline-inducible promoter. In some embodiments, the inducible promoter is a doxycycline-inducible promoter. In other embodiments, a promoter is a constitutive promoter (active in vivo, unregulated).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

Vectors used for delivery of an engineered polynucleotide include minicircles, plasmids, bacterial artificial chromosomes (BACs), and yeast artificial chromosomes. Transposon-based systems, such as the piggyBac™ system (e.g., Chen et al. Nature Communications. 2020; 11 (1): 3446), is also contemplated herein.

A pluripotent stem cells, in some embodiments, comprises an engineered polynucleotide comprising an open reading frame encoding a protein selected from ZNF281, LHX8, and SOHLH1. In some embodiments, the engineered polynucleotide comprises an open reading frame encoding ZNF281. In some embodiments, the engineered polynucleotide comprises an open reading frame encoding LHX8. In some embodiments, the engineered polynucleotide comprises an open reading frame encoding SOHLH1.

In some embodiments, a pluripotent stem cell comprises an engineered polynucleotide comprising an open reading frame encoding ZNF281 and an engineered polynucleotide comprising an open reading frame encoding LHX8. In some embodiments, a pluripotent stem cell comprises an engineered polynucleotide comprising an open reading frame encoding ZNF281 and an engineered polynucleotide comprising an open reading frame encoding SOHLH1. In some embodiments, a pluripotent stem cell comprises an engineered polynucleotide comprising an open reading frame encoding LHX8 and an engineered polynucleotide comprising an open reading frame encoding SOHLH1. In some embodiments, a pluripotent stem cell comprises an engineered polynucleotide comprising an open reading frame encoding ZNF281, an engineered polynucleotide comprising an open reading frame encoding LHX8, and an engineered polynucleotide comprising an open reading frame encoding SOHLH1.

An engineered polynucleotide encoding comprising an open reading frame encoding Zinc Finger Protein 281 (ZNF281) (e.g., UniprotKB Accession No. Q9Y2X9), in some embodiments, encodes a protein comprising the sequence of:

(SEQ ID NO: 1)
MKIGSGFLSGGGGTGSSGGSGSGGGGSGGGGGGGSSGRRAEMEPTFPQGM
VMFNHRLPPVTSFTRPAGSAAPPPQCVLSSSTSAAPAAEPPPPPAPDMTF
KKEPAASAAAFPSQRTSWGFLQSLVSIKQEKPADPEEQQSHHHHHHHHYG
GLFAGAEERSPGLGGGEGGSHGVIQDLSILHQHVQQQPAQHHRDVLLSSS
SRTDDHHGTEEPKQDTNVKKAKRPKPESQGIKAKRKPSASSKPSLVGDGE
GAILSPSQKPHICDHCSAAFRSSYHLRRHVLIHTGERPFQCSQCSMGFIQ
KYLLQRHEKIHSREKPFGCDQCSMKFIQKYHMERHKRTHSGEKPYKCDTC
QQYFSRTDRLLKHRRTCGEVIVKGATSAEPGSSNHTNMGNLAVLSQGNTS
SSRRKTKSKSIAIENKEQKTGKINESQISNNINMQSYSVEMPTVSSSGGI
IGTGIDELQKRVPKLIFKKGSRKNTDKNYLNFVSPLPDIVGQKSLSGKPS
GSLGIVSNNSVETIGLLQSTSGKQGQISSNYDDAMQFSKKRRYLPTASSN
SAFSINVGHMVSQQSVIQSAGVSVLDNEAPLSLIDSSALNAEIKSCHDKS
GIPDEVLQSILDQYSNKSESQKEDPFNIAEPRVDLHTSGEHSELVQEENL
SPGTQTPSNDKASMLQEYSKYLQQAFEKSTNASFTLGHGFQFVSLSSPLH
NHTLFPEKQIYTTSPLECGFGQSVTSVLPSSLPKPPFGMLFGSQPGLYLS
ALDATHQQLTPSQELDDLIDSQKNLETSSAFQSSSQKLTSQKEQKNLESS
TGFQIPSQELASQIDPQKDIEPRTTYQIENFAQAFGSQFKSGSRVPMTFI
TNSNGEVDHRVRTSVSDFSGYTNMMSDVSEPCSTRVKTPTSQSYR

An engineered polynucleotide encoding comprising an open reading frame encoding LIM Homeobox 8 (LHX8) (e.g., UniprotKB Accession No. Q68G74), in some embodiments, encodes a protein comprising the sequence of:

(SEQ ID NO: 2)
MQILSRCQGLMSEECGRTTALAAGRTRKGAGEEGLVSPEGAGDEDSCSSS
APLSPSSSPRSMASGSGCPPGKCVCNSCGLEIVDKYLLKVNDLCWHVRCL
SCSVCRTSLGRHTSCYIKDKDIFCKLDYFRRYGTRCSRCGRHIHSTDWVR
RAKGNVYHLACFACFSCKRQLSTGEEFALVEEKVLCRVHYDCMLDNLKRE
VENGNGISVEGALLTEQDVNHPKPAKRARTSFTADQLQVMQAQFAQDNNP
DAQTLQKLAERTGLSRRVIQVWFQNCRARHKKHVSPNHSSSTPVTAVPPS
RLSPPMLEEMAYSAYVPQDGTMLTALHSYMDAHSPTTLGLQPLLPHSMTQ
LPISHT

An engineered polynucleotide encoding comprising an open reading frame encoding Spermatogenesis and Oogenesis Specific Basic Helix-Loop-Helix 1 (SOHLH1) (e.g., UniprotKB Accession No. Q6IUP1), in some embodiments, encodes a protein comprising the sequence of:

(SEQ ID NO: 3)
MASGGHERANEDYRVSGITGCSKTPQPETQDSLQTSSQSSALCTAPVAAA
NLGPSLRRNVVSERERRRRISLSCEHLRALLPQFDGRREDMASVLEMSVY
FLQLAHSMDPSWEQLSVPQPPQEMWHMWQGDVLQVTLANQIADSKPDSGI
AKPSAVSRVQDPPCFGMLDTDQSQATERESELLERPSSCPGHRQSALSFS
EPESSSLGPGLPPWIPHSWQPATPEASDIVPGGSHQVASLAGDPESSGML
AEEANLVLASVPDARYTTGAGSDVVDGAPFLMTTNPDWWLGSVEGRGGPA
LARSSPVDGAEPSFIGDPELCSQELQAGPGELWGLDFGSPGLALKDEADS
IFPDFFP

A pluripotent stem cells, in some embodiments, further comprises an engineered polynucleotide comprising an open reading frame encoding a protein selected from FIGLA, DLX5, and HHEX. In some embodiments, the engineered polynucleotide comprises an open reading frame encoding FIGLA. In some embodiments, the engineered polynucleotide comprises an open reading frame encoding DLX5. In some embodiments, the engineered polynucleotide comprises an open reading frame encoding HHEX.

In some embodiments, a pluripotent stem cell comprises an engineered polynucleotide comprising an open reading frame encoding FIGLA and an engineered polynucleotide comprising an open reading frame encoding DLX5. In some embodiments, a pluripotent stem cell comprises an engineered polynucleotide comprising an open reading frame encoding FIGLA and an engineered polynucleotide comprising an open reading frame encoding HHEX. In some embodiments, a pluripotent stem cell comprises an engineered polynucleotide comprising an open reading frame encoding DLX5 and an engineered polynucleotide comprising an open reading frame encoding HHEX. In some embodiments, a pluripotent stem cell comprises an engineered polynucleotide comprising an open reading frame encoding FIGLA, an engineered polynucleotide comprising an open reading frame encoding DLX5, and an engineered polynucleotide comprising an open reading frame encoding HHEX.

An engineered polynucleotide encoding comprising an open reading frame encoding Folliculogenesis Specific BHLH Transcription Factor (FIGLA) (e.g., UniprotKB Accession No. Q6QHK4), in some embodiments, encodes a protein comprising the sequence of:

(SEQ ID NO: 4)
MDPAPGVLDPRAAPPALLGTPQAEVLEDVLREQFGPLPQLAAVCRLKRLP
SGGYSSTENLQLVLERRRVANAKERERIKNLNRGFARLKALVPFLPQSRK
PSKVDILKGATEYIQVLSDLLEGAKDSKKQDPDEQSYSNNSSESHISSAR
QLSRNITQHISCAFGLKNEEEGPWADGGSGEPAHACRHSVMSTTEIISPT
RSLDRFPEVELLSHRLPQV

An engineered polynucleotide encoding comprising an open reading frame encoding Distal-Less Homeobox 5 (DLX5) (e.g., UniprotKB Accession No. P56178), in some embodiments, encodes a protein comprising the sequence of:

(SEQ ID NO: 5)
MTGVFDRRVPSIRSGDFQAPFQTSAAMHHPSQESPTLPESSATDSDYYSP
TGGAPHGYCSPTSASYGKALNPYQYQYHGVNGSAGSYPAKAYADYSYASS
YHQYGGAYNRVPSATNQPEKEVTEPEVRMVNGKPKKVRKPRTIYSSFQLA
ALQRRFQKTQYLALPERAELAASLGLTQTQVKIWFQNKRSKIKKIMKNGE
MPPEHSPSSSDPMACNSPQSPAVWEPQGSSRSLSHHPHAHPPTSNQSPAS
SYLENSASWYTSAASSINSHLPPPGSLQHPLALASGTLY

An engineered polynucleotide encoding comprising an open reading frame encoding Hematopoietically Expressed Homeobox (HHEX) (e.g., UniprotKB Accession No. Q03014), in some embodiments, encodes a protein comprising the sequence of:

(SEQ ID NO: 6)
MQYPHPGPAAGAVGVPLYAPTPLLQPAHPTPFYIEDILGRGPAAPTPAPT
LPSPNSSFTSLVSPYRTPVYEPTPIHPAFSHHSAAALAAAYGPGGFGGPL
YPFPRTVNDYTHALLRHDPLGKPLLWSPFLQRPLHKRKGGQVRFSNDQTI
ELEKKFETQKYLSPPERKRLAKMLQLSERQVKTWFQNRRAKWRRLKQENP
QSNKKEELESLDSSCDQRQDLPSEQNKGASLDSSQCSPSPASQEDLESEI
SEDSDQEVDIEGDKSYFNAG

A pluripotent stem cells, in some embodiments, further comprises an engineered polynucleotide comprising an open reading frame encoding a protein selected from DDX4, DAZL, and BOLL. In some embodiments, the engineered polynucleotide comprises an open reading frame encoding DDX4. In some embodiments, the engineered polynucleotide comprises an open reading frame encoding DAZL. In some embodiments, the engineered polynucleotide comprises an open reading frame encoding BOLL.

In some embodiments, a pluripotent stem cell comprises an engineered polynucleotide comprising an open reading frame encoding DDX4 and an engineered polynucleotide comprising an open reading frame encoding DAZL. In some embodiments, a pluripotent stem cell comprises an engineered polynucleotide comprising an open reading frame encoding DDX4 and an engineered polynucleotide comprising an open reading frame encoding BOLL. In some embodiments, a pluripotent stem cell comprises an engineered polynucleotide comprising an open reading frame encoding DAZL and an engineered polynucleotide comprising an open reading frame encoding BOLL. In some embodiments, a pluripotent stem cell comprises an engineered polynucleotide comprising an open reading frame encoding DDX4, an engineered polynucleotide comprising an open reading frame encoding DAZL, and an engineered polynucleotide comprising an open reading frame encoding BOLL.

An engineered polynucleotide encoding comprising an open reading frame encoding DEAD-Box Polypeptide-4 (DDX4) (e.g., UniprotKB Accession No. Q9NQI0), in some embodiments, encodes a protein comprising the sequence of:

(SEQ ID NO: 7)
MGDEDWEAEINPHMSSYVPIFEKDRYSGENGDNFNRTPASSSEMDDGPSR
RDHFMKSGFASGRNFGNRDAGECNKRDNTSTMGGFGVGKSFGNRGFSNSR
FEDGDSSGFWRESSNDCEDNPTRNRGFSKRGGYRDGNNSEASGPYRRGGR
GSFRGCRGGFGLGSPNNDLDPDECMQRTGGLFGSRRPVLSGTGNGDTSQS
RSGSGSERGGYKGLNEEVITGSGKNSWKSEAEGGESSDTQGPKVTYIPPP
PPEDEDSIFAHYQTGINFDKYDTILVEVSGHDAPPAILTFEEANLCQTLN
NNIAKAGYTKLTPVQKYSIPIILAGRDLMACAQTGSGKTAAFLLPILAHM
MHDGITASRFKELQEPECIIVAPTRELVNQIYLEARKFSFGTCVRAVVIY
GGTQLGHSIRQIVQGCNILCATPGRLMDIIGKEKIGLKQIKYLVLDEADR
MLDMGFGPEMKKLISCPGMPSKEQRQTLMFSATFPEEIQRLAAEFLKSNY
LFVAVGQVGGACRDVQQTVLQVGQFSKREKLVEILRNIGDERTMVFVETK
KKADFIATFLCQEKISTTSIHGDREQREREQALGDFRFGKCPVLVATSVA
ARGLDIENVQHVINFDLPSTIDEYVHRIGRTGRCGNTGRAISFFDLESDN
HLAQPLVKVLTDAQQDVPAWLEEIAFSTYIPGFSGSTRGNVFASVDTRKG
KSTLNTAGFSSSQAPNPVDDESWD

An engineered polynucleotide encoding comprising an open reading frame encoding Deleted in AZoospermia (DAZL) (e.g., UniprotKB Accession No. Q92904), in some embodiments, encodes a protein comprising the sequence of:

(SEQ ID NO: 8)
MSTANPETPNSTISREASTQSSSAATSQGYILPEGKIMPNTVFVGGIDVR
MDETEIRSFFARYGSVKEVKIITDRTGVSKGYGFVSFFNDVDVQKIVESQ
INFHGKKLKLGPAIRKQNLCAYHVQPRPLVFNHPPPPQFQNVWTNPNTET
YMQPTTTMNPITQYVQAYPTYPNSPVQVITGYQLPVYNYQMPPQWPVGEQ
RSYVVPPAYSAVNYHCNEVDPGAEVVPNECSVHEATPPSGNGPQKKSVDR
SIQTVVSCLFNPENRLRNSVVTQDDYFKDKRVHHFRRSRAMLKSV

An engineered polynucleotide encoding comprising an open reading frame encoding Boule Homolog (BOLL) (e.g., UniprotKB Accession No. Q8N9W6), in some embodiments, encodes a protein comprising the sequence of:

(SEQ ID NO: 9)
MQTDSLSPSPNPVSPVPLNNPTSAPRYGTVIPNRIFVGGIDFKTNESDLR
KFFSQYGSVKEVKIVNDRAGVSKGYGFVTFETQEDAQKILQEAEKLNYKD
KKLNIGPAIRKQQVGIPRSSIMPAAGTMYLTTSTGYPYTYHNGVAYFHTP
EVTSVPPPWPSRSVCSSPVMVAQPIYQQPAYHYQATTQYLPGQWQWSVPQ
PSASSAPFLYLQPSEVIYQPVEIAQDGGCVPPPLSLMETSVPEPYSDHGV
QATYHQVYAPSAITMPAPVMQPEPIKTVWSIHY

The number of copies of an engineered polynucleotide delivered to a PSC may vary. In some embodiments, a PSC comprises 1-20 copies of an engineered polynucleotide. For example, and PSC may comprise 1-15, 1-10, 2-10, 2-15, 2-10, 5-20, 5-15, or 5-10 copies of an engineered polynucleotide. In some embodiments, a PSC comprises 8-10 copies of an engineered polynucleotide. Greater than 20 copies are also contemplated herein.

### Methods of Producing Oogonia—Like Cells

The methods of producing oogonia-like cells provided herein, in some aspects, comprises culturing, in culture media, a population of pluripotent stem cells (PSCs) to produce an expanded population of PSCs; and expressing in PSCs of the expanded population a protein selected from ZNF281, LHX8, and SOHLH1 to produce oogonia-like cells.

In some embodiments, the PSCs of the expanded population comprise an engineered polynucleotide comprising an open reading frame encoding ZNF281. In some embodiments, the PSCs of the expanded population comprise an engineered polynucleotide comprising an open reading frame encoding LHX8. In some embodiments, the PSCs of the expanded population comprise an engineered polynucleotide comprising an open reading frame encoding SOHLH1. In some embodiments, the PSCs of the expanded population further comprise an engineered polynucleotide comprising an open reading frame encoding a FIGLA protein. In some embodiments, the PSCs of the expanded population further comprise: an engineered polynucleotide comprising an open reading frame encoding a DLX5 protein; and an engineered polynucleotide comprising an open reading frame encoding an HHEX protein. In some embodiments, the PSCs of the expanded population further comprise: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein.

In some embodiments, the open reading frame of the engineered polynucleotide is operably linked to a heterologous promoter.

In some embodiments, the heterologous promoter is an inducible promoter, non-limiting examples of which are provided elsewhere herein.

The population a starting population comprises about 1×10²-1×10¹⁰, about 1×10²-1×10⁹, about 1×10²-1×10⁸, or about 1×10²-1×10⁷ PSCs. In some embodiments, the population comprises about 1×10³-1×10⁸ or about 1×10³-1×10⁷ PSCs. In some embodiments, the population comprises about 1×10⁴-1×10⁷ or about 1×10⁵-1×10⁶ PSCs. In some embodiments, the population comprises about 1×10¹ PSCs, about 1×10² PSCs, about 1×10³ PSCs, about 1×10⁴ PSCs, about 1×10⁵ PSCs, about 1×10⁶ PSCs, about 1×10⁷ PSCs, about 1×10⁸ PSCs, about 1×10⁹ PSCs, or about 1×10¹⁰ PSCs.

In some embodiments, the population of PSCs is cultured for about 2 to about 6 days, about 2 to about 5 days, about 2 to about 4 days, about 3 to about 6 days, about 3 to about 5 days, or about 3 to about 4 days. In some embodiments, the population of PSCs is cultured for about 2 days, about 3 days, about 4 days, about 5 days, or about 6 days.

Some methods of the present disclosure provide methods comprising (a) delivering to PSCs an engineered polynucleotide comprising an inducible promoter operably linked to an open reading frame encoding a protein selected from ZNF281, LHX8, and SOHLH1 (or selected from ZNF281, LHX8, SOHLH1, FIGLA, DLX5, HHEX, DDX4, DAZL, and BOLL); (b) culturing the PSCs in feeder-free, serum-free culture media to produce an expanded population of PSCs; and (c) culturing PSCs of the expanded population in a series of induction media comprising an inducing agent to produce DDX4+ oogonia-like cells. In some embodiments, the series of induction media comprises a first, a second, a third, and a fourth induction media.

In some embodiments, the PSCs are cultured in feeder-free, serum-free culture media for about 6 to about 24 hours. For example, the PSC may be cultured in feeder-free, serum-free culture media for about, 6 to about 12 hours. In some embodiments, the PSCs are cultured in feeder-free, serum-free culture media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the expanded population of PSCs comprises at least 5×10³ PSCs. For example, the expanded population (e.g., at the time of induction) may comprise at least 1×10⁴, at least 1×10⁵, at least 1×10⁶, or at least 1×10⁷ PSCs. In some embodiments, the expanded population of PSCs comprises about 5×10³ PSCs to about 1×10⁷ PSCs.

In some embodiments, PSCs of the expanded population are cultured at a density of about 2,000 cells/cm² to about 3,000 cells/cm². In some embodiments, PSCs of the expanded population are cultured at a density of about 500/cm²-10000/cm² PSCs. In some embodiments, the PSCs of the expanded population are cultured at a density of about 1000/cm²-9500/cm² PSCs. In some embodiments, PSCs of the expanded population are cultured at a density of about 1500/cm²-9000/cm² PSCs. In some embodiments, PSCs of the expanded population are cultured at a density of about 2000/cm²-8500/cm² PSCs. In some embodiments, PSCs of the expanded population are cultured at a density of about 2500/cm²-8000/cm² PSCs. In some embodiments, PSCs of the expanded population are cultured at a density of about 3000/cm²-7500/cm² PSCs. In some embodiments, PSCs of the expanded population are cultured at a density of about 3500/cm²-7000/cm² PSCs. In some embodiments, the population comprises 4000/cm²-6500/cm² PSCs. In some embodiments, PSCs of the expanded population are cultured at a density of about 4500/cm²-6000/cm² PSCs. In some embodiments, PSCs of the expanded population are cultured at a density of about 5000/cm²-5500/cm² PSCs. In some embodiments, PSCs of the expanded population are cultured at a density of at least 500/cm² PSCs, at least 1000/cm² PSCs, at least 1500/cm² PSCs, at least 2000/cm² PSCs, at least 2500/cm² PSCs, at least 3000/cm² PSCs, at least 3500/cm² PSCs, at least 4000/cm² PSCs, at least 4500/cm² PSCs, at least 5000/cm² PSCs, at least 5500/cm² PSCs, at least 6000/cm² PSCs, at least 6500/cm² PSCs, at least 7000/cm² PSCs, at least 7500/cm² PSCs, at least 8000/cm² PSCs, at least 8500/cm² PSCs, at least 9000/cm² PSCs, at least 9500/cm² PSCs, or at least 10000/cm² PSCs.

In some embodiments, PSCs of the expanded population are cultured for no longer than 8 days, no longer than 7 days, no longer than 6 days, no longer than 5 days, or no longer than 4 days. For example, PSCs of the expanded population may be cultured for about 2 to about 8 days, about 2 to about 7 days, about 2 to about 6 days, about 2 to about 5 days, about 2 to about 4 days, about 3 to about 8 days, about 3 to about 7 days, about 3 to about 6 days, about 3 to about 5 days, or about 3 to about 4 days. In some embodiments, PSCs of the expanded population are cultured for about 2 days, about 3 days, about 4 days, about 5 days, about 6 days, about 7 days, or about 8 days.

In some embodiments, PSCs of the expanded population are cultured in a first induction media for about 6 to about 36 hours. For example, the PSC may be cultured in a first induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a first induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a second induction media for about 6 to about 36 hours. For example, the PSC may be cultured in a second induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a second induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a third induction media for about 6 to about 36 hours. For example, the PSC may be cultured in a third induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a third induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs of the expanded population are cultured in a fourth induction media for about 6 to about 36 hours. For example, the PSC may be cultured in a fourth induction media for about 6 to about 24 hours, about 6 to about 18 hours, about 6 to about 12 hours, 12 to about 36 hours, about 12 to about 24 hours, about 12 to about 18 hours, 18 to about 36 hours, or about 18 to about 24 hours. In some embodiments, the PSCs are cultured in a fourth induction media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours.

In some embodiments, PSCs are incubated for at least 6 hours. In some embodiments, after incubation, the media is removed from the plate and the plate is washed with DMEM/F12. In some embodiments, Media #1 (Table 1) is added to the plate at a volume of 250 μL/cm². In some embodiments, the iPSCs are incubated for 18 hours before Media #1 is removed and Media #2 is added (Table 1). In some embodiments, the iPSCs are incubated for 24 hours before Media #2 is removed and Media #3 is added (Table 1). In some embodiments, the iPSCs are incubated for 18 hours before Media #3 is removed and Media #4 is added (Table 1). In some embodiments, after the iPSCs are incubated with Media #4, DDX4+ oogonia-like cells are present in the plate.

### Transfection Methods

The engineered polynucleotide of the present disclosure may be delivered to a PSC using any one or more transfection method, including chemical transfection methods, viral transduction methods, and electroporation.

In some embodiments, an engineered polynucleotide is delivered on a vector. A vector is any vehicle, for example, a virus or a plasmid, that is used to transfer a desired polynucleotide into a host cell, such as a PSC. In some embodiments, the vector is a viral vector. In some embodiments, a viral vector is not a naturally occurring viral vector. The viral vector may be from adeno-associated virus (AAV), adenovirus, herpes simplex virus, lentiviral, retrovirus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B 19. Other viral vectors are encompassed by the present disclosure.

In some embodiments, a viral vector is an AAV vector. AAV is a small, non-enveloped virus that packages a single-stranded linear DNA genome that is approximately 5 kb long and has been adapted for use as a gene transfer vehicle (Samulski, R J et al., Annu Rev Virol. 2014; 1 (1): 427-51). The coding regions of AAV are flanked by inverted terminal repeats (ITRs), which act as the origins for DNA replication and serve as the primary packaging signal (McLaughlin, S K et al. Virol. 1988; 62 (6): 1963-73; Hauswirth, W W et al. 1977; 78 (2): 488-99). Thus, an AAV vector typically includes ITR sequences. Both positive and negative strands are packaged into virions equally well and capable of infection (Zhong, L et al. Mol Ther. 2008; 16 (2): 290-5; Zhou, X et al. Mol Ther. 2008; 16 (3): 494-9; Samulski, R J et al. Virol. 1987; 61 (10): 3096-101). In addition, a small deletion in one of the two ITRs allows packaging of self-complementary vectors, in which the genome self-anneals after viral uncoating. This results in more efficient transduction of cells but reduces the coding capacity by half (McCarty, D M et al. Mol Ther. 2008; 16 (10): 1648-56; McCarty, D M et al. Gene Ther. 2001; 8 (16): 1248-54).

In some embodiments, a polynucleotide is delivered to a cell using a transposon/transposase system. For example, the piggyBac™ transposon system may be used. A piggyBac™ transposon is a mobile genetic element that efficiently transposes between vectors and chromosomes via a “cut and paste” mechanism (Woodard et al. 2015). During transposition, the piggyBac™ transposase recognizes transposon-specific inverted terminal repeat sequences (ITRs) located on both ends of the transposon vector and efficiently moves the contents from the original sites and integrates them into TTAA chromosomal sites. The piggyBac™ transposon system facilitates efficient integration of a polynucleotide into a cell genome.

Thus, in some embodiments, the method further comprises delivering to a PSC a transposon comprising an engineered polynucleotide and also delivering a transposase.

In some embodiments, an engineered polynucleotide is delivered to a cell using electroporation. Electroporation is a physical transfection method that uses an electrical pulse to create temporary pores in cell membranes through which the engineered polynucleotide can pass into cells. See, e.g., Chicaybam L et al. Front. Bioeng. Biotechnol., 23 Jan. 2017.

Following transfection, the engineered polynucleotides may be integrated into the genome of a PSC. In some embodiments, an engineered polynucleotide may further comprise an antibiotic resistance gene to confer resistance to an antibiotic used in an antibiotic drug selection process. In this way, a ‘pure’ population of cells comprising an integrated engineered polynucleotide may be obtained. In some embodiments, a population of cells comprising an integrated engineered polynucleotide are selected using antibiotic drug selection. Antibiotic drug selection is the process of treating a population of cells with an antibiotic so that only cells that are capable of surviving in the presence of said antibiotic will remain in the population. Non-limiting examples of antibiotics that may be used for antibiotic drug selection include: puromycin, blasticidin, geneticin, hygromycin, mycophenolic acid, zeocin, carbenicillin, kanemycin, ampicillin, and actinomycin.

### Culture Media

The methods provided herein, in some embodiments, comprise culturing PSCs in a feeder-free, serum-free culture media. Culture media may comprise, for example, a solubilized basement membrane preparation extracted from the Engelbreth-Holm-Swarm (EHS) mouse sarcoma (e.g., Corning® Matrigel® Matrix) (coated at 75 to 150 μl per cm² of lot-based diluted suspension). In some embodiments, the solubilized basement membrane preparation comprises one or more extracellular matrix (ECM) protein and one or more growth factor. For example, the ECM proteins may be selected from Laminin, Collagen IV, heparan sulfate proteoglycans, and entactin/nidogen.

In some embodiments, culture media further comprises one or more growth factor, for example, selected from recombinant human basic fibroblast growth factor (rh bFGF) (e.g., 80 ng/ml to 120 ng/ml) and recombinant human transforming growth factor β (rh TGFβ) (e.g., 20 to 25 pM). In some embodiments, culture media further comprises rh bFGF and rh TGFβ. In some embodiments, culture media comprises mTeSR™ media (STEMCELL Technologies).

In some embodiments, a first induction media comprises one or more of (e.g., 2, 3, 4, or more of) B-27 Supplement (e.g., 90× to 110×), L-alanyl-L-glutamine (e.g., 1.8 mM to 2.2 mM), an inducing agent (e.g., doxycycline (e.g., 50 ng/ml to 2000 ng/ml)), Activin A (e.g., 50 ng/ml to 150 ng/ml), a glycogen synthase kinase (GSK) 3 inhibitor (e.g., 2.8 μM to 3.2 μM), a selective FGFR1 and FGFR3 inhibitor (e.g., 90 nM to 110 nM), and a small molecule ROCK inhibitor (e.g., 8 μM to 12 μM). In some embodiments, a first induction media comprises B-27, L-alanyl-L-glutamine, an inducing agent (e.g., doxycycline), Activin A, a glycogen synthase kinase (GSK) 3 inhibitor, and a selective FGFR1 and FGFR3 inhibitor. For example, the first induction media may comprise aRB27 Media, doxycycline, Activin A, CHIR99021, and PD 173074.

In some embodiments, the second induction media comprises one or more of (e.g., 2, 3, 4, or more of) B-27 Supplement (e.g., 90× to 110×), an inducing agent (e.g., doxycycline (e.g., 50 ng/ml to 2000 ng/ml)), a small molecule inhibitor of tankyrase (TNKS) (e.g., 0.9 μM to 1.1 μM), and a human bone morphogenic protein 4 (hBMP4) (e.g., 20 ng/ml to 250 ng/ml). In some embodiments, the second induction media comprises B-27, an inducing agent (e.g., doxycycline), a small molecule inhibitor of tankyrase (TNKS), and a human bone morphogenic protein 4 (hBMP4). For example, the second induction media may comprise aRB27 Media, doxycycline, XAV939, and human bone morphogenic protein 4 (hBMP4).

In some embodiments, the third induction media comprises one or more of (e.g., 2, 3, 4, or more of) B-27, an inducing agent (e.g., doxycycline), a small molecule inhibitor of tankyrase (e.g., 0.9 μM to 1.1 μM), stem cell factor (SCF) (e.g., 25 ng/ml to 200 ng/ml), and epidermal growth factor (EGF) (e.g., 25 ng/ml to 100 ng/ml). In some embodiments, the third induction media comprises B-27 Supplement (e.g., 90× to 110×), an inducing agent (e.g., doxycycline (e.g., 50 ng/ml to 2000 ng/ml)), a small molecule inhibitor of tankyrase (e.g., 0.9 μM to 1.1 μM), stem cell factor (SCF) (e.g., 25 ng/ml to 200 ng/ml), and epidermal growth factor (EGF) (e.g., 25 ng/ml to 100 ng/ml). For example, the third induction media may comprise aRB27 Media, doxycycline, XAV939, SCF, and EGF.

In some embodiments, the fourth induction media comprises one or more of (e.g., 2, 3, 4, or more of) B-27 Supplement (90-110×), an inducing agent (e.g., doxycycline (e.g., 50 ng/ml to 2000 ng/ml)), a small molecule inhibitor of tankyrase (e.g., 0.9 μM to 1.1 μM), hBMP4 (e.g., 20 ng/ml to 250 ng/ml), SCF (e.g., 25 ng/ml to 200 ng/ml), and EGF (e.g., 25 ng/ml to 100 ng/ml). In some embodiments, the fourth induction media comprises B-27, an inducing agent (e.g., doxycycline), a small molecule inhibitor of tankyrase, hBMP4, SCF, and EGF. For example, the fourth induction media may comprise aRB27 Media, doxycycline, XAV939, hBMP4, SCF, and EGF.

The ‘aRB27 Media’ used herein comprises Advanced RPMI, B-27™ Supplement, minus vitamin A (Thermo Fisher) or plus vitamin A, GlutaMAX™ Supplement (Thermo Fisher), non-essential amino acids (NEAA), Primocin® (a broad-spectrum antibiotic), and Y-27632 (a small molecule ROCK inhibitor).

GlutaMAX™ Supplement comprises L-alanyl-L-glutamine, which is a dipeptide substitute for L-glutamine.

Activin-A is a dimeric glycoprotein, which belongs to the transforming growth factor-β (TGF-β) family.

CHIR99021 is an aminopyrimidine derivative that is an extremely potent glycogen synthase kinase (GSK) 3 inhibitor, inhibiting both GSK3β (IC₅₀=6.7 nM) and GSK3a (IC₅₀=10 nM). GSK3 is a serine/threonine kinase that is a key inhibitor of the WNT pathway; therefore, CHIR99021 functions as a WNT activator.

PD 173074 is a selective FGFR1 and FGFR3 inhibitor (IC₅₀ values are ˜5 nM, ˜21.5 nM, ˜100 nM, ˜17600 nM and ˜19800 nM for FGFR3, FGFR1, VEGFR2, PDGFR and c-Src respectively, and >50000 nM for EGFR, InsR, MEK and PKC).

XAV939 is a potent, small molecule inhibitor of tankyrase (TNKS) 1 and 2 (IC₅₀=11 and 4 nM, respectively) (Huang et al.). By inhibiting TNKS activity, XAV939 increases the protein levels of the axin-GSK3β complex and promotes the degradation of β-catenin in SW480 cells (Huang et al.), thereby inhibiting WNT pathway downstream actions.

### Therapeutic Compositions and Method of Use

The present disclosure provides, in some embodiments, therapeutic compositions comprising the oogonia-like cells produced herein. In some embodiments, the compositions further comprise a pharmaceutically-acceptable excipient. The compositions, in some embodiments, are cryopreserved.

Such compositions may be administered to a subject, such as a human subject, using any suitable route of administration. Suitable routes of administration include, for example, parenteral routes such as intravenous, intrathecal, parenchymal, or intraventricular routes. Suitable routes of administration include, for example, parenteral routes such as intravenous, intrathecal, parenchymal, or intraventricular injection.

In some embodiments, a subject is a human subject. The subject may have an infertility disorder, such as Turner syndrome. Infertility disorders include disorders in which germ cell development is severely affected in a female fetus. Blood sample analysis may be used to diagnose an infertility disorder. In some embodiments, the infertility disorder may include damage to oogonia.

The compositions may be administered to a subject in a therapeutically effective amount. The term ““therapeutically effective amount” refers to the amount of oogonia required to confer therapeutic effect on a subject, either alone or in combination with at least one other active agent. Effective amounts vary, as recognized by those skilled in the art, depending on the route of administration, excipient usage, and co-usage with other active agents. The quantity to be administered depends on the subject to be treated, including, for example, the strength of an individual's immune system or genetic predispositions. Suitable dosage ranges are readily determinable by one skilled in the art and may be on the order of micrograms of the polypeptide of this disclosure. The dosage of the preparations disclosed herein may depend on the route of administration and varies according to the size of the subject.

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### Additional Embodiments

The disclosure also relates to the additional embodiments set out in the numbered paragraphs below:
1. A pluripotent stem cell (PSC) comprising: an engineered polynucleotide comprising an open reading frame encoding a protein selected from Zinc Finger Protein 281 (ZNF281), LIM Homeobox 8 (LHX8), and Spermatogenesis and Oogenesis Specific Basic Helix-Loop-Helix 1 (SOHLH1).
2. The PSC of paragraph 1, wherein the PSC comprises the engineered polynucleotide comprising an open reading frame encoding ZNF281.
3. The PSC of paragraph 1 or 2, wherein the PSC comprises the engineered polynucleotide comprising an open reading frame encoding LHX8.
4. The PSC of any one of the preceding paragraphs, wherein the PSC comprises the engineered polynucleotide comprising an open reading frame encoding SOHLH1.
5. The PSC of any one of the preceding paragraphs, wherein the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.
6. The PSC of any one of the preceding paragraphs, wherein PSC further comprises an engineered polynucleotide comprising an open reading frame encoding a Folliculogenesis Specific BHLH Transcription Factor (FIGLA) protein, optionally wherein the PSC expresses or overexpresses FIGLA.
7. The PSC of paragraph 6, wherein the PSC further comprises: an engineered polynucleotide comprising an open reading frame encoding a Distal-Less Homeobox 5 (DLX5) protein; and an engineered polynucleotide comprising an open reading frame encoding an Hematopoietically Expressed Homeobox (HHEX) protein, optionally wherein the PSC expresses or overexpresses the DLX5 protein and the HHEX protein.
8. The PSC of paragraph 7, wherein the PSC further comprises: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein, optionally wherein the PSC expresses or overexpresses the DDX4 protein, the DAZL protein, and the BOLL protein.
9. The PSC of any one of the preceding paragraphs, wherein the open reading frame of the engineered polynucleotide is operably linked to a heterologous promoter.
10. The PSC of paragraph 9, wherein the heterologous promoter is an inducible promoter.
11. A pluripotent stem cell (PSC) comprising: a protein selected from ZNF281, LHX8, and SOHLH1, wherein the protein is overexpressed.
12. The PSC of paragraph 11, wherein the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.
13. The PSC of paragraph 12, wherein the PSC further comprises a FIGLA protein, optionally wherein the PSC expresses or overexpresses FIGLA.
14. The PSC of paragraph 13, wherein the PSC further comprises: a DLX5 protein and an HHEX protein, optionally wherein the PSC expresses or overexpresses the DLX5 protein and the HHEX protein.
15. The PSC of paragraph 14, wherein the PSC further comprises: a DDX4 protein, a DAZL protein, and a BOLL protein, optionally wherein the PSC expresses or overexpresses the DDX4 protein, the DAZL protein, and the BOLL protein.
16. The PSC of any one of the preceding paragraphs, wherein the PSC is a human PSC.
17. The PSC of any one of the preceding paragraphs, wherein the PSC is an induced PSC (iPSC).
18. The PSC of any one of the preceding paragraphs, wherein the PSC comprises 1-20, optionally 8-10, copies of the engineered polynucleotide comprising the open reading frame encoding the protein selected from ZNF281, LHX8, and SOHLH1.
19. A composition comprising: a population of the PSC of any one of the preceding paragraphs or described elsewhere herein.
20. The composition of paragraph 18, wherein the population comprises at least 2500/cm² of the PSC.
21. A method, comprising: culturing, in culture media, a population of pluripotent stem cells (PSCs) to produce an expanded population of PSCs; and expressing in PSCs of the expanded population a protein selected from ZNF281, LHX8, and SOHLH1 to produce oogonia-like cells.
22. The method of paragraph 21, wherein the PSCs of the expanded population comprise an engineered polynucleotide comprising an open reading frame encoding ZNF281.
23. The method of paragraph 21 or 22, wherein the PSCs of the expanded population comprise an engineered polynucleotide comprising an open reading frame encoding LHX8.
24. The method of any one of paragraphs 21-23, wherein the PSCs of the expanded population comprise an engineered polynucleotide comprising an open reading frame encoding SOHLH1.
25. The method of any one of paragraph 21-24, wherein the PSCs of the expanded population further comprise an engineered polynucleotide comprising an open reading frame encoding a FIGLA protein.
26. The method of paragraph 25, wherein the PSCs of the expanded population further comprise: an engineered polynucleotide comprising an open reading frame encoding a DLX5 protein; and an engineered polynucleotide comprising an open reading frame encoding an HHEX protein.
27. The method of paragraph 26, wherein the PSCs of the expanded population further comprise: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein.
28. The method of any one of the preceding paragraphs, wherein the open reading frame of the engineered polynucleotide is operably linked to a heterologous promoter.
29. The method of any one of the preceding paragraphs, wherein the heterologous promoter is an inducible promoter.
30. The method of any one of the preceding paragraphs, wherein the population comprises 1×10²-1×10⁷ PSCs.
31. The method of any one of the preceding paragraphs, wherein the population of PSCs is cultured for about 3-5 days, optionally about 4 days.
32. The method of paragraph 31, wherein the population of PSCs is cultured for about days.
33. The method of any one of the preceding paragraphs, wherein the oogonia-like cells are DDX4⁺.
34. The method of any one of the preceding paragraphs, wherein the oogonia-like cells independently have a diameter of about 20 micrometers to 180 micrometers.
35. A method comprising:
(a) delivering to pluripotent stem cells (PSCs) an engineered polynucleotide comprising an inducible promoter operably linked to an open reading frame encoding a protein selected from ZNF281, LHX8, and SOHLH1;
(b) culturing the PSCs in feeder-free, serum-free culture media to produce an expanded population of PSCs; and
(c) culturing PSCs of the expanded population in a series of induction media comprising an inducing agent to produce DDX4+ oogonia-like cells.
36. The method of paragraph 35, wherein the engineered polynucleotide is a transposon and the delivering further comprises delivering a transposase to the PSCs.
37. The method of paragraph 35 or 36, wherein the inducible promoter is a chemically-inducible promoter, optionally a doxycycline-inducible promoter.
38. The method of any one of paragraphs 35-37, wherein the feeder-free, serum-free culture media of (b) comprises a solubilized basement membrane preparation extracted from the Engelbreth-Holm-Swarm (EHS) mouse sarcoma.
39. The method of paragraph 38, wherein the solubilized basement membrane preparation comprises extracellular matrix (ECM) proteins and growth factors.
40. The method of paragraph 39, wherein the ECM proteins are selected from Laminin, Collagen IV, heparan sulfate proteoglycans, and entactin/nidogen.
41. The method of any one of paragraphs 35-40, wherein the feeder-free, serum-free culture media of (b) comprises growth factors selected from recombinant human basic fibroblast growth factor (rh bFGF) and recombinant human transforming growth factor β (rh TGFβ).
42. The method of any one of paragraphs 35-41, wherein the culturing of (b) is for about 6 to about 24 hours.
43. The method of any one of paragraphs 35-42, wherein the PSCs of the expanded population of (c) are cultured at a density of about 2,000 cells/cm² to about 3,000 cells/cm².
44. The method of any one of paragraphs 35-43, wherein the culturing of (c) comprises culturing the PSCs is a first induction media, culturing the PSCs in a second induction media, culturing the PSCs in a third induction media, and culturing the PSCs in a fourth induction media.
45. The method of paragraph 44, wherein the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.
46. The method of paragraph 44 or 45, wherein the second induction media comprises one or more of B-27, an inducing agent (e.g., doxycycline), a small molecule inhibitor of tankyrase (TNKS), and a human bone morphogenic protein 4 (hBMP4).
47. The method of any one of paragraphs 44-46, wherein the third induction media comprises one or more of B-27, an inducing agent (e.g., doxycycline), a small molecule inhibitor of tankyrase, stem cell factor (SCF), and epidermal growth factor (EGF).
48. The method of any one of paragraphs 44-47, wherein the fourth induction media comprises one or more of B-27, an inducing agent (e.g., doxycycline), a small molecule inhibitor of tankyrase, hBMP4, SCF, and EGF.
49. An oogonia-like cell produced by the method of any one of the preceding paragraphs.

### EXAMPLES

### Example 1. A Three Transcription Factor Cocktail to Induce DDX4+ Oogonia-Like Cells from hiPSCs

Current methods to generate DDX4+ human oogonia-like cells from human induced pluripotent stem cells (hiPSCs) are laborious, expensive and time consuming. For example, one such methodology utilizes co-cultures of induced human primordial germ cell-like cells (hPGCLCs) and mouse E12.5 fetal gonad cells (mFGCs) to form xenogeneic reconstituted ovaries (xrOvaries), which when grown over approximately 120 days induce a small population of human DDX4+ oogonia-like cells (Yamashiro et al., Science. 2018 Oct. 19; 362 (6412): 356-360). This method, while able to generate oogonia-like cells, require extensive rodent handling expertise, expensive culturing techniques, and lengthy differentiation periods. This Example describes an efficient and reliable method of generating DDX4+ oogonia-like cells from hiPSCs in four days using direct transcription factor (TF) overexpression in conjunction with growth factor culturing.

The present disclosure relates to the identification of three transcription factors (ZNF281, LHX8, SOHLH1) that, when overexpressed individually or in combination, drive formation of DDX4+ oogonia-like cells from hiPSCs.

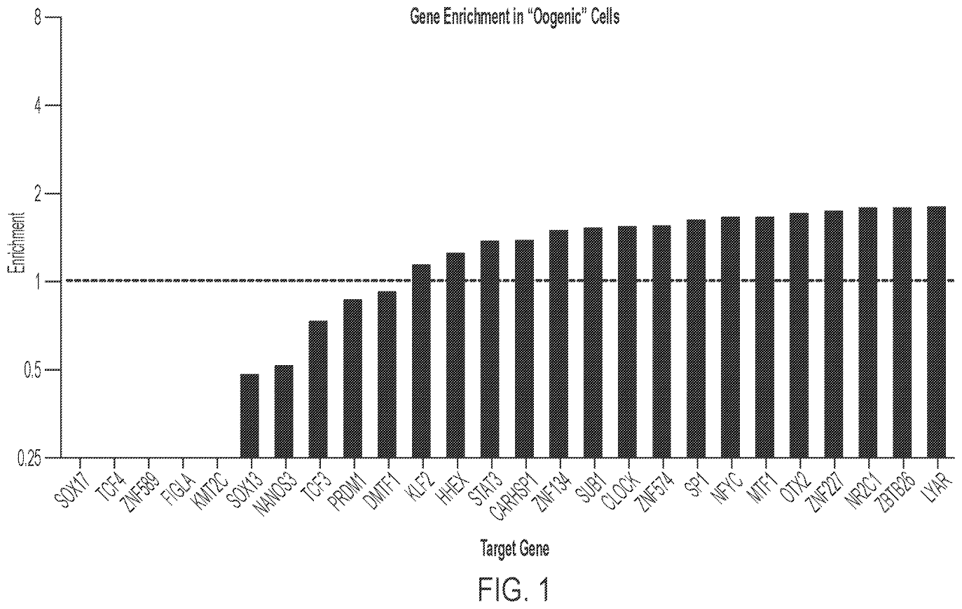

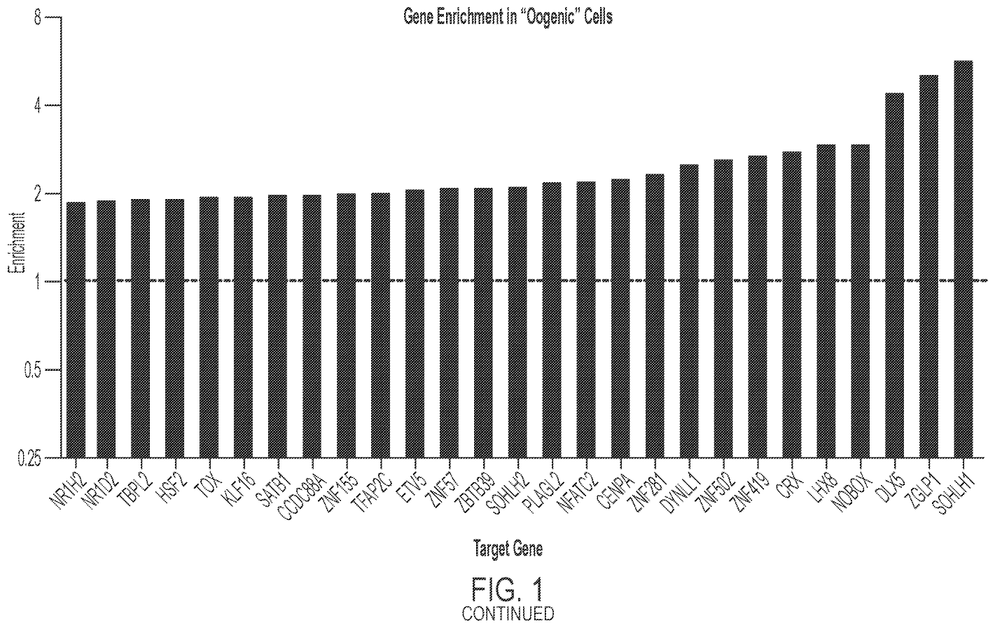

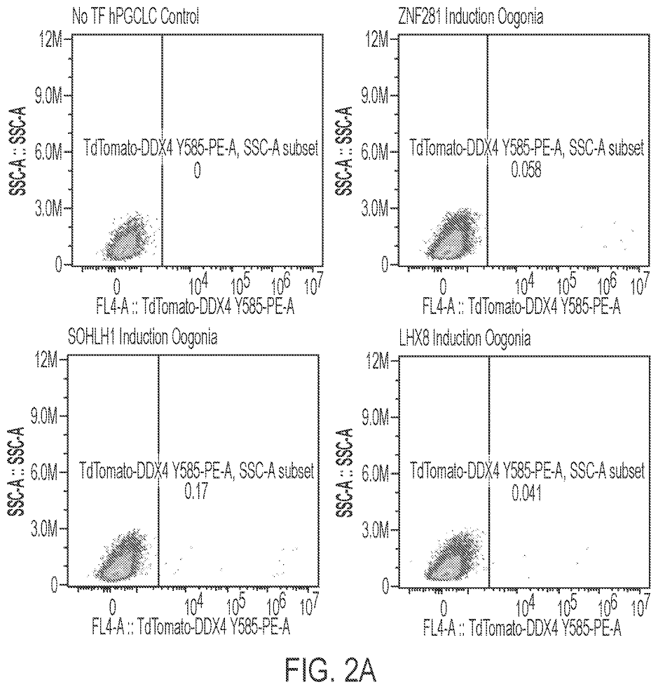

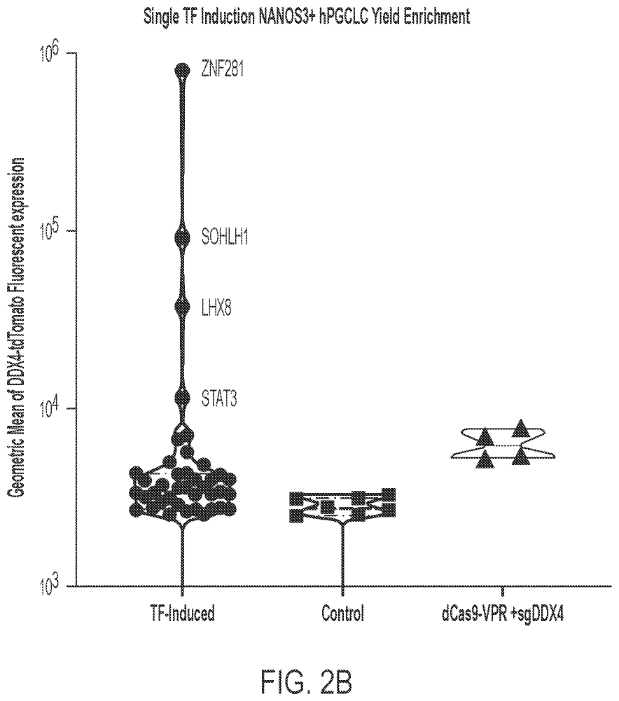

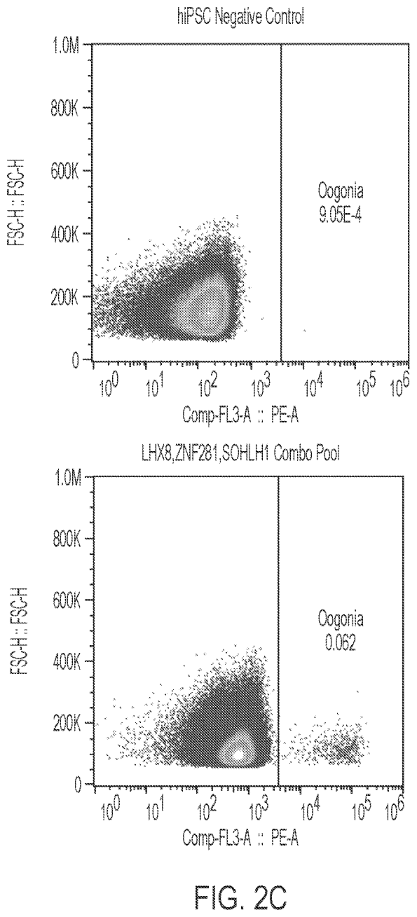

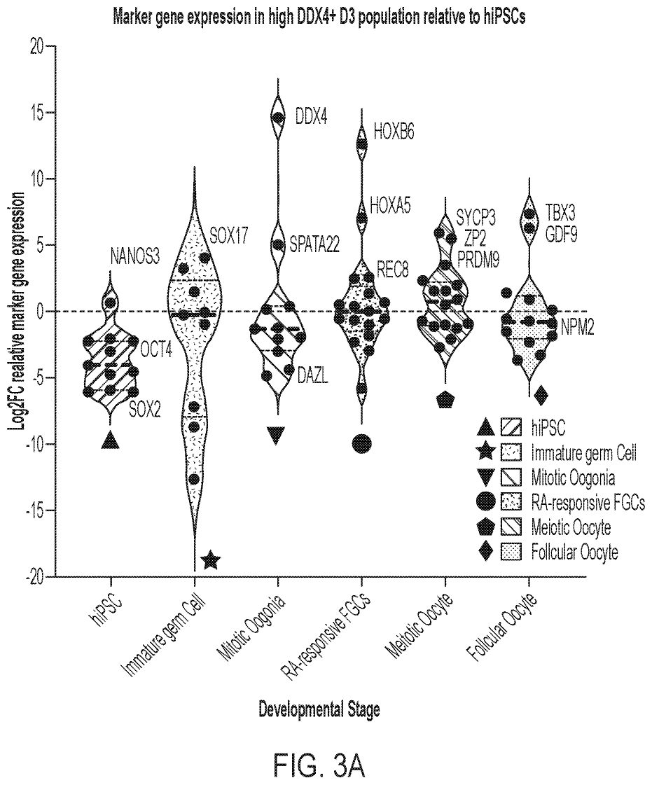

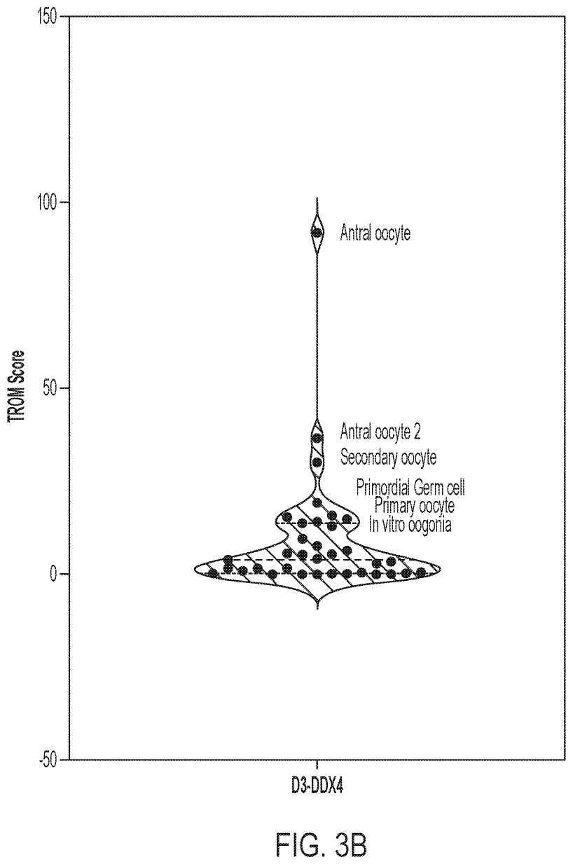

To identify the three transcription factors (ZNF281, LHX8, and SOHLH1), a computational algorithm was developed to predict transcription factors that participate in human oocyte differentiation (Kramme et al. Cell Reports Methods, 100082, 2021). Utilizing a predicted set of 53 transcription factors, 53 individual hiPSC lines harboring one TF each as well as combinatorial pools of TFs spanning 42 million unique combinations were generated. Through TF induction followed by bulk RNA sequencing (RNA-seq) and single cell RNA-Seq, a subset of TFs was identified that drives oogenic gene expression signatures (FIG. 1). Next, a DDX4-tdTomato hiPSC reporter line was utilized, which induced human germ cell (hPGCLC) formation using cytokines in conjunction with individual TF overexpression. Three TFs (ZNF281, LHX8, and SOHLH1) were identified that showed production a small population of high DDX4 expressing cells when individually overexpressed in hiPSCs (FIGS. 2A-2B). Next, a combination hiPSC line harboring integrated expression vectors of all three TFs was built, termed DDX4-3 (D3), which when induced during hPGCLC formation drove a higher yield of DDX4+ cells than the individual TF expressions (FIG. 2C). This high DDX4+ population, driven by the three TFs, was characterized using bulk RNA-Seq and showed that overexpression of the three TFs in combination drives upregulation of key oocyte-regulating genes (FIG. 3A). Through transcriptomic comparison to a human ovarian atlas reference, it was shown that the D3 combination drives potent oocyte-like transcriptomic signatures, as was observed in the statistically significant TROM classification when compared to secondary and antral oocytes (FIG. 3B). Taken together, these results demonstrate that direct overexpression of these three TFs individually and in combinations in conjunction with hPGCLC differentiation is sufficient to generate DDX4+ oogonia-like cells from hiPSCs in four days.

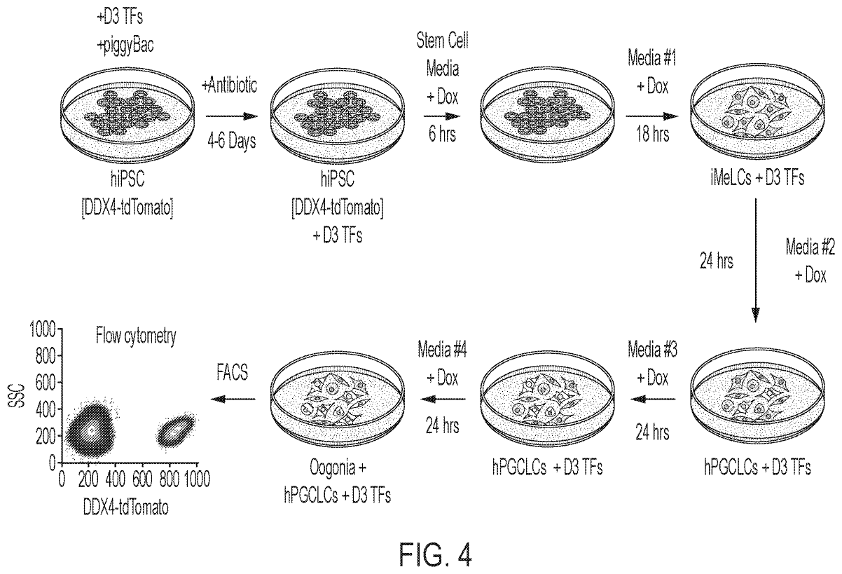

This TF-based high DDX4-expressing cell differentiation protocol is highly scalable and cost effective (FIG. 4). Briefly, the TFs were individually or in combination integrated into female hiPSCs as doxycycline inducible gene expression cassettes using the piggyBac transposase system. Antibiotic drug selection was used to achieve a pure population of cells containing integrated expression cassettes. The TF-containing hiPSCs were then grown feeder-free on Corning® Matrigel® Matrix the serum-free culturing mTeSR™ medium.

To induce oogonia formation, the hiPSCs were seeded at a density of 2,500 cells/cm² on Corning® Matrigel® Matrix coated plates in mTeSR™ media plus ˜8-10 μM Y-27623 and 0.5-3 μg/mL doxycycline. After about six hours, the media was removed and the plate was briefly washed with DMEM/F12. The media was then replaced with an incipient mesoderm induction media (Media #1) listed in Table 1. After about 18 hours in Media #1, the media was removed and the plate was again briefly washed with DMEM/F12 and the hPGCLC induction media was added (Media #2 listed in Table 1) for 24 hours. After 24 hours, the media was replaced by Media #3 and again incubated for 24 hours. Finally, Media #3 was replaced with Media #4 and incubated for 24 hours, after which high DDX4-expressing cells were present in the cell pool. At this point, DDX4+ expressing cells were isolated using sorting techniques and used for downstream analysis.

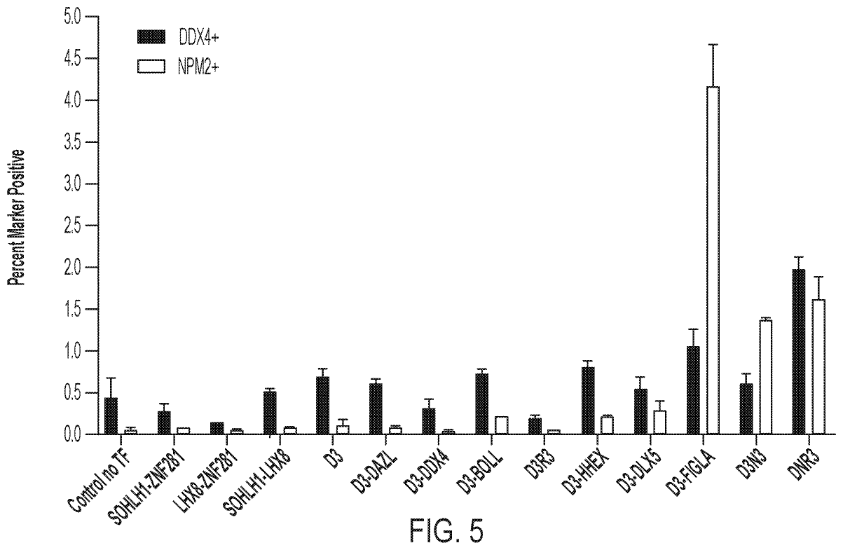

FIG. 5 shows testing of combinations of TFs for oogonia formation. The percentage of cells that are express DDX4 and the percentage of cells that express NPM2 are shown. For oogonia-induction, individual independent expression of SOHLH1, ZNF281, or LHX8 (referred to herein as D3 or D3 combo) all show yield of DDX4+ cells. Combinatorial overexpression of all three shows 100-1000 fold increase in efficiency of DDX4+ oogonia production. Combinations of two, particularly combinations with LHX8 present show 10-50 fold increase compared to individual overexpression. Additionally, addition of FIGLA to the D3 combo increases yield 10 fold compared to D3 alone, and also induced NPM2+ oocyte-like formation. Similarly, it was found that addition of DLX5, HHEX and FIGLA to the D3 combination to show increased DDX4+ yield compared to D3 alone, a combination referred to herein as D3N3. Finally, additional overexpression of DDX4, DAZL, and BOLL with the D3N3 combination showed increase in DDX4+ yield compared to D3 alone, a combination referred to herein as DNR3. D3+HHEX, D3+DLX5, D3+DAZL, D3+DDX4, D3+BOLL, and D3+DAZL, DDX4, BOLL showed no increase in DDX4+ yield compared to D3 alone.

### Methods and Materials

iPSC Culture

iPSCs were cultured in mTESR 1 medium (Stemcell Technologies) on standard polystyrene plates coated with hESC-qualified Corning® Matrigel® Matrix. Medium was changed daily. Passaging was performed with TRYPLE (Gibco). iPSCs were treated with ˜8-12 μM Y-27632 (Ambeed) for 24 hours after each passage. Mycoplasma testing was performed by PCR every 3 months; all cells tested negative.

### TF Plasmid Construction

TF cDNAs were synthesized as full-length transcripts or obtained from the ORFeome (The ORFeome Colobration, Nat Methods. 13, 191-192 (2016)) as Gateway entry clones. These were cloned into a doxycycline-inducible PiggyBac expression plasmid (Addgene #175503) using MegaGate (Kramme et al., STAR Protoc. 2, 100907 (2021)). The final expression constructs were verified by Sanger sequencing, which also served to determine the barcode sequence for each TF.

TF Plasmid Integration into hiPSCs

Expression plasmids containing TF cDNAs under the control of a doxycycline-inducible promoter were integrated into iPSCs using PiggyBac transposase. To perform the integration, ˜50-100 fmol of TF cDNA plasmid, ˜150-250 ng PiggyBac transposase expression plasmid, and ˜100,000-200,000 iPSCs were combined in Lonza P3 buffer and electroporated using a Lonza Nucleofector 4D. After electroporation, cells were seeded in 24-well plates in mTeSR™ Plus Medium+˜8-12 μM Y-27632. Selection with the appropriate agent (typically puromycin) commenced 48 hours after electroporation and continued for ˜3-5 days. Cells were then passaged without drug selectin for ˜3 days to allow for non-integrated plasmid loss. Finally, cells were again passaged under drug selection to generate a pure, selected integrant pool. Presence and approximate copy number of integrated TF plasmids was confirmed by qPCR on genomic DNA. For oogonia, pools of hiPSCs were utilized, not single cell selected clones. Average copy number was 8-10.

### Protocol for Oogonia Induction Via TF Overexpression

hiPSCs containing integrated TF expression plasmids were cultured in mTeSR™ medium on Corning® Matrigel® Matrix. For induction in monolayer, hiPSCs were disassociated to single cells using StemPro™ Accutase™ Cell Dissociation Reagent and seeded on Corning® Matrigel® Matrix or vitronectin XF coated plates at a density of 2,500-3,000 cells/cm² in mTeSR™ media+˜8-10 μM Y-27632 and ˜0.5-3 μg/ml doxycycline for about 6 hours. Media was then removed and washed with dPBS or DMEM/F12 and replaced with aRB27 media #1. After about 12-18 hours of induction, media #1 was removed and washed with dPBS or DMEM/F12 and replaced by media #2. After about 24 hours, media #2 was removed and replaced by media #3. After about 24 hours, media #3 was replaced with media #4. Oogonia was harvested for use after about 24 hours in media #4 or additionally after about two further days of culture in media #4 (at day 6 of the protocol). Oogonia-like cells were isolated via a DDX4 reporter. Oogonia can additionally be generated via embryoid formation through methods established in Yamashiro et al. Science, 362 (6412), 356-360, Kobayashi et al., Stem Cell Reports, 9 (3), 999-1015, Stem Cell Reports, 9 (3), 999-1015, Murase et al., The EMBO Journal, 1-25, 2020 and Mitsunaga et al., Proceedings of the National Academy of Sciences of the United States of America, 114 (46), E9913-E9922 with similar efficiency compared to monolayer protocols.

### Protocol for Expansion of Oogonia-Like Cells Post-Isolation

Expansion of sorted oogonia-like cells can be performed via FACS isolation of DDX4+ cells from step 6 at about day 4 or 6. Isolated oogonia cells are seeded onto Corning® Matrigel® Matrix coated plates in S-CM media described in Kobayashi et al. 2022 with Y-27632 and doxycycline. Half media is changed every 2-3 days and cells can be expanded, passaged and re-purified as necessary.

TABLE 1
Media compositions used in Example 1

| aRB27 Media | Media #1 | Media #2 | Media #3 | Media #4 |
|---|---|---|---|---|
| Advanced | aRB27 Media | aRB27 Media | aRB27 Media | aRB27 Media |
| RPMI | | | | |
| B27 Minus or | Doxycycline | Doxycycline | Doxycycline | Doxycycline |
| Plus Vitamin A | (~0.5-3 μl/mL) | (~0.5-3 μl/mL) | (~0.5-3 μl/mL) | (~0.5-3 μl/mL) |
| (100X) | | | | |
| GlutaMAX ™ | Activin A | XAV939 | XAV939 | XAV939 |
| Supplement | (~50-150 ng/mL) | (~0.5-3 μM) | (~0.5-3 μM) | (~0.5-3 μM) |
| (100X) | | | | |
| Non-essential | Chiron99021 | hBMP4 | SCF | hBMP4 |
| amino acids | (~2-4 μM) | (~35-45 ng/mL) | (~50-150 ng/mL) | (~35-45 ng/mL) |
| (NEAA) (100X) | | | | |
| Primocin | PD173074 | | EGF | SCF |
| (100X)* | (~50-150 nM) | | (~25-75 ng/mL) | (~50-150 ng/mL) |
| Y-27632 | | | | EGF |
| (~8-12 μM) | | | | (~25-75 ng/mL) |

*Optional antibiotic

TABLE 2
Cytokines used in Example 1

| | | Product | | |
|---|---|---|---|---|
| Cytokines | Supplier | Number | Lot Number | Buffer |
| Activin A | Peprotech | 120-14P | #1117605 | 0.1% BSA |
| | | | | in water |
| BMP4 | R&D | 314-BP- | #BEM12220081 | 0.1% BSA in |
| | Systems | 010 | | 4 mM HCl |
| EGF | Peprotech | AF-100- | #0820AFC05 | 5% trehalose |
| | | 15-1MG | | (or 0.1% BSA) |
| | | | | in water |
| SCF | Peprotech | 300-07- | #111834-1 | 0.1% BSA |
| | | 250UG | | in water |

TABLE 3
Inhibitors used in Example 1

| | | Lot | |
|---|---|---|---|
| Inhibitors | Supplier | Number | Solvent |
| CHIR99021 | Axon | 12 | 66% H2O, 33% DMSO |
| (hydrochloride) | Medchem | | |
| PD173074 | Selleck | S126404 | DMSO |
| XAV939 | Tocris | 5B/251235 | DMSO |
| Y-27632 | Ambeed | Various | H2O |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A pluripotent stem cell (PSC) comprising: an engineered polynucleotide comprising an open reading frame encoding a protein selected from Zinc Finger Protein 281 (ZNF281), LIM Homeobox 8 (LHX8), and Spermatogenesis and Oogenesis Specific Basic Helix-Loop-Helix 1 (SOHLH1).

2. The PSC of claim 1, wherein the PSC comprises the engineered polynucleotide comprising an open reading frame encoding ZNF281.

3. The PSC of claim 1, wherein the PSC comprises the engineered polynucleotide comprising an open reading frame encoding LHX8.

4. The PSC of claim 1, wherein the PSC comprises the engineered polynucleotide comprising an open reading frame encoding SOHLH1.

5. The PSC of claim 1, wherein the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.

6. The PSC of claim 1, wherein PSC further comprises an engineered polynucleotide comprising an open reading frame encoding a Folliculogenesis Specific BHLH Transcription Factor (FIGLA) protein, optionally wherein the PSC expresses or overexpresses FIGLA.

7. The PSC of claim 6, wherein the PSC further comprises: an engineered polynucleotide comprising an open reading frame encoding a Distal-Less Homeobox 5 (DLX5) protein; and an engineered polynucleotide comprising an open reading frame encoding an Hematopoietically Expressed Homeobox (HHEX) protein, optionally wherein the PSC expresses or overexpresses the DLX5 protein and the HHEX protein.

8. The PSC of claim 7, wherein the PSC further comprises: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein, optionally wherein the PSC expresses or overexpresses the DDX4 protein, the DAZL protein, and the BOLL protein.

9. The PSC of claim 1, wherein the open reading frame of the engineered polynucleotide is operably linked to a heterologous promoter.

10. The PSC of claim 9, wherein the heterologous promoter is an inducible promoter.

11. A pluripotent stem cell (PSC) comprising: a protein selected from ZNF281, LHX8, and SOHLH1, wherein the protein is overexpressed.

12. The PSC of claim 11, wherein the PSC expresses or overexpresses: ZNF281; LHX8; SOHLH1; ZNF281 and LHX8; ZNF281 and SOHLH1; LHX8 and SOHLH1; or ZNF281, LHX8, and SOHLH1.

13. The PSC of claim 12, wherein the PSC further comprises a FIGLA protein, optionally wherein the PSC expresses or overexpresses FIGLA.

14. The PSC of claim 13, wherein the PSC further comprises: a DLX5 protein and an HHEX protein, optionally wherein the PSC expresses or overexpresses the DLX5 protein and the HHEX protein.

15. The PSC of claim 14, wherein the PSC further comprises: a DDX4 protein, a DAZL protein, and a BOLL protein, optionally wherein the PSC expresses or overexpresses the DDX4 protein, the DAZL protein, and the BOLL protein.

16. The PSC of claim 1, wherein the PSC is a human PSC.

17. The PSC of claim 1, wherein the PSC is an induced PSC (iPSC).

18. The PSC of claim 1, wherein the PSC comprises 1-20, optionally 8-10, copies of the engineered polynucleotide comprising the open reading frame encoding the protein selected from ZNF281, LHX8, and SOHLH1.

19. A composition comprising: a population of the PSC of claim 1.

20. The composition of claim 18, wherein the population comprises at least 2500/cm² of the PSC.

21. A method, comprising: culturing, in culture media, a population of pluripotent stem cells (PSCs) to produce an expanded population of PSCs; and expressing in PSCs of the expanded population a protein selected from ZNF281, LHX8, and SOHLH1 to produce oogonia-like cells.

22. The method of claim 21, wherein the PSCs of the expanded population comprise an engineered polynucleotide comprising an open reading frame encoding ZNF281.

23. The method of claim 21, wherein the PSCs of the expanded population comprise an engineered polynucleotide comprising an open reading frame encoding LHX8.

24. The method of claim 21, wherein the PSCs of the expanded population comprise an engineered polynucleotide comprising an open reading frame encoding SOHLH1.

25. The method of claim 21, wherein the PSCs of the expanded population further comprise an engineered polynucleotide comprising an open reading frame encoding a FIGLA protein.

26. The method of claim 25, wherein the PSCs of the expanded population further comprise: an engineered polynucleotide comprising an open reading frame encoding a DLX5 protein; and an engineered polynucleotide comprising an open reading frame encoding an HHEX protein.

27. The method of claim 26, wherein the PSCs of the expanded population further comprise: an engineered polynucleotide comprising an open reading frame encoding a DDX4 protein; an engineered polynucleotide comprising an open reading frame encoding a DAZL protein; and an engineered polynucleotide comprising an open reading frame encoding a BOLL protein.

28. The method of claim 21, wherein the open reading frame of the engineered polynucleotide is operably linked to a heterologous promoter.

29. The method of claim 21, wherein the heterologous promoter is an inducible promoter.

30. The method of claim 21, wherein the population comprises 1×10²-1×10⁷ PSCs.

31. The method of claim 21, wherein the population of PSCs is cultured for about 3-5 days, optionally about 4 days.

32. The method of claim 31, wherein the population of PSCs is cultured for about days.

33. The method of claim 21, wherein the oogonia-like cells are DDX4⁺.

34. The method of claim 21, wherein the oogonia-like cells independently have a diameter of about 20 micrometers to 180 micrometers.

35. A method comprising:
(a) delivering to pluripotent stem cells (PSCs) an engineered polynucleotide comprising an inducible promoter operably linked to an open reading frame encoding a protein selected from ZNF281, LHX8, and SOHLH1;
(b) culturing the PSCs in feeder-free, serum-free culture media to produce an expanded population of PSCs; and
(c) culturing PSCs of the expanded population in a series of induction media comprising an inducing agent to produce DDX4+ oogonia-like cells.

36. The method of claim 35, wherein the engineered polynucleotide is a transposon and the delivering further comprises delivering a transposase to the PSCs.

37. The method of claim 35, wherein the inducible promoter is a chemically-inducible promoter, optionally a doxycycline-inducible promoter.

38. The method of claim 35, wherein the feeder-free, serum-free culture media of (b) comprises a solubilized basement membrane preparation extracted from the Engelbreth-Holm-Swarm (EHS) mouse sarcoma.

39. The method of claim 38, wherein the solubilized basement membrane preparation comprises extracellular matrix (ECM) proteins and growth factors.

40. The method of claim 39, wherein the ECM proteins are selected from Laminin, Collagen IV, heparan sulfate proteoglycans, and entactin/nidogen.

41. The method of claim 35, wherein the feeder-free, serum-free culture media of (b) comprises growth factors selected from recombinant human basic fibroblast growth factor (rh bFGF) and recombinant human transforming growth factor β (rh TGFβ).

42. The method of claim 35, wherein the culturing of (b) is for about 6 to about 24 hours.

43. The method of claim 35, wherein the PSCs of the expanded population of (c) are cultured at a density of about 2,000 cells/cm² to about 3,000 cells/cm².

44. The method of claim 35, wherein the culturing of (c) comprises culturing the PSCs is a first induction media, culturing the PSCs in a second induction media, culturing the PSCs in a third induction media, and culturing the PSCs in a fourth induction media.

45. The method of claim 44, wherein the first induction media comprises one or more of B-27, L-alanyl-L-glutamine, an inducing agent (e.g., doxycycline), Activin A, a glycogen synthase kinase (GSK) 3 inhibitor, and a selective FGFR1 and FGFR3 inhibitor.

46. The method of claim 44, wherein the second induction media comprises one or more of B-27, an inducing agent (e.g., doxycycline), a small molecule inhibitor of tankyrase (TNKS), and a human bone morphogenic protein 4 (hBMP4).

47. The method of claim 44, wherein the third induction media comprises one or more of B-27, an inducing agent (e.g., doxycycline), a small molecule inhibitor of tankyrase, stem cell factor (SCF), and epidermal growth factor (EGF).

48. The method of claim 44, wherein the fourth induction media comprises one or more of B-27, an inducing agent (e.g., doxycycline), a small molecule inhibitor of tankyrase, hBMP4, SCF, and EGF.

49. An oogonia-like cell produced by the method of claim 35.


SEQUENCE LISTING (9 sequences)

SEQ ID NO: 1 - protein, 895 aa, synthetic construct
MKIGSGFLSGGGGTGSSGGSGSGGGGSGGGGGGGSSGRRAEMEPTFPQGMVMFNHRLPPVTSFTRPAGSAAPPPQCVLSSSTSAAPAAEPPPPPAPDMTFKKEPAASAAAFPSQRTSWGFLQSLVSIKQEKPADPEEQQSHHHHHHHHYGGLFAGAEERSPGLGGGEGGSHGVIQDLSILHQHVQQQPAQHHRDVLLSSSSRTDDHHGTEEPKQDTNVKKAKRPKPESQGIKAKRKPSASSKPSLVGDGEGAILSPSQKPHICDHCSAAFRSSYHLRRHVLIHTGERPFQCSQCSMGFIQKYLLQRHEKIHSREKPFGCDQCSMKFIQKYHMERHKRTHSGEKPYKCDTCQQYFSRTDRLLKHRRTCGEVIVKGATSAEPGSSNHTNMGNLAVLSQGNTSSSRRKTKSKSIAIENKEQKTGKTNESQISNNINMQSYSVEMPTVSSSGGIIGTGIDELQKRVPKLIFKKGSRKNTDKNYLNFVSPLPDIVGQKSLSGKPSGSLGIVSNNSVETIGLLQSTSGKQGQISSNYDDAMQFSKKRRYLPTASSNSAFSINVGHMVSQQSVIQSAGVSVLDNEAPLSLIDSSALNAEIKSCHDKSGIPDEVLQSILDQYSNKSESQKEDPFNIAEPRVDLHTSGEHSELVQEENLSPGTQTPSNDKASMLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 2 - protein, 356 aa, synthetic construct
MQILSRCQGLMSEECGRTTALAAGRTRKGAGEEGLVSPEGAGDEDSCSSSAPLSPSSSPRSMASGSGCPPGKCVCNSCGLEIVDKYLLKVNDLCWHVRCLSCSVCRTSLGRHTSCYIKDKDIFCKLDYFRRYGTRCSRCGRHIHSTDWVRRAKGNVYHLACFACFSCKRQLSTGEEFALVEEKVLCRVHYDCMLDNLKREVENGNGISVEGALLTEQDVNHPKPAKRARTSFTADQLQVMQAQFAQDNNPDAQTLQKLAERTGLSRRVIQVWFQNCRARHKKHVSPNHSSSTPVTAVPPSRLSPPMLEEMAYSAYVPQDGTMLTALHSYMDAHSPTTLGLQPLLPHSMTQLPISHT

SEQ ID NO: 3 - protein, 357 aa, synthetic construct
MASGGHERANEDYRVSGITGCSKTPQPETQDSLQTSSQSSALCTAPVAAANLGPSLRRNVVSERERRRRISLSCEHLRALLPQFDGRREDMASVLEMSVYFLQLAHSMDPSWEQLSVPQPPQEMWHMWQGDVLQVTLANQIADSKPDSGIAKPSAVSRVQDPPCFGMLDTDQSQATERESELLERPSSCPGHRQSALSFSEPESSSLGPGLPPWIPHSWQPATPEASDIVPGGSHQVASLAGDPESSGMLAEEANLVLASVPDARYTTGAGSDVVDGAPFLMTTNPDWWLGSVEGRGGPALARSSPVDGAEPSFIGDPELCSQELQAGPGELWGLDFGSPGLALKDEADSIFPDFFP

SEQ ID NO: 4 - protein, 219 aa, synthetic construct
MDPAPGVLDPRAAPPALLGTPQAEVLEDVLREQFGPLPQLAAVCRLKRLPSGGYSSTENLQLVLERRRVANAKERERIKNLNRGFARLKALVPFLPQSRKPSKVDILKGATEYIQVLSDLLEGAKDSKKQDPDEQSYSNNSSESHTSSARQLSRNITQHISCAFGLKNEEEGPWADGGSGEPAHACRHSVMSTTEIISPTRSLDRFPEVELLSHRLPQV

SEQ ID NO: 5 - protein, 289 aa, synthetic construct
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 6 - protein, 270 aa, synthetic construct
MQYPHPGPAAGAVGVPLYAPTPLLQPAHPTPFYIEDILGRGPAAPTPAPTLPSPNSSFTSLVSPYRTPVYEPTPIHPAFSHHSAAALAAAYGPGGFGGPLYPFPRTVNDYTHALLRHDPLGKPLLWSPFLQRPLHKRKGGQVRFSNDQTIELEKKFETQKYLSPPERKRLAKMLQLSERQVKTWFQNRRAKWRRLKQENPQSNKKEELESLDSSCDQRQDLPSEQNKGASLDSSQCSPSPASQEDLESEISEDSDQEVDIEGDKSYFNAG

SEQ ID NO: 7 - protein, 724 aa, synthetic construct
MGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVTYIPPPPPEDEDSIFAHYQTGINFDKYDTILVEVSGHDAPPAILTFEEANLCQTLNNNIAKAGYTKLTPVQKYSIPIILAGRDLMACAQTGSGKTAAFLLPILAHMMHDGITASRFKELQEPECIIVAPTRELVNQIYLEARKFSFGTCVRAVVIYGGTQLGHSIRQIVQGCNILCATPGRLMDIIGKEKIGLKQIKYLVLDEADRMLDMGFGPEMKKLISCPGMPSKEQRQTLMFSATFPEEIQRLAAEFLKSNYLFVAVGQVGGACRDVQQTVLQVGQFSKREKLVEILRNIGDERTMVFVETKKKADFIATFLCQEKISTTSIHGDREQREREQALGDFRFGKCPVLVATSVAARGLDIENVQHVINFDLPSTIDEYVHRIGRTGRCGNTGRAISFFDLESDNHLAQPLVKVLTDAQQDVPAWLEEIAFSTYIPGFSGSTRGNVFASVDTRKGKSTLNTAGFSSSQAPNPVDDESWD

SEQ ID NO: 8 - protein, 295 aa, synthetic construct
MSTANPETPNSTISREASTQSSSAATSQGYILPEGKIMPNTVFVGGIDVRMDETEIRSFFARYGSVKEVKIITDRTGVSKGYGFVSFFNDVDVQKIVESQINFHGKKLKLGPAIRKQNLCAYHVQPRPLVFNHPPPPQFQNVWTNPNTETYMQPTTTMNPITQYVQAYPTYPNSPVQVITGYQLPVYNYQMPPQWPVGEQRSYVVPPAYSAVNYHCNEVDPGAEVVPNECSVHEATPPSGNGPQKKSVDRSIQTVVSCLFNPENRLRNSVVTQDDYFKDKRVHHFRRSRAMLKSV

SEQ ID NO: 9 - protein, 283 aa, synthetic construct
MQTDSLSPSPNPVSPVPLNNPTSAPRYGTVIPNRIFVGGIDFKTNESDLRKFFSQYGSVKEVKIVNDRAGVSKGYGFVTFETQEDAQKILQEAEKLNYKDKKLNIGPAIRKQQVGIPRSSIMPAAGTMYLTTSTGYPYTYHNGVAYFHTPEVTSVPPPWPSRSVCSSPVMVAQPIYQQPAYHYQATTQYLPGQWQWSVPQPSASSAPFLYLQPSEVIYQPVEIAQDGGCVPPPLSLMETSVPEPYSDHGVQATYHQVYAPSAITMPAPVMQPEPIKTVWSIHY